(19) United States
(12) Patent Application Publication
(10) Pub. No.: US 2020/0155196 A1
(43) Pub. Date: May 21, 2020

(54) SURGICAL ACCESS SYSTEMS

(71) Applicant: MEDROBOTICS CORPORATION, Raynham, MA (US)

(72) Inventors: Ian Darisse, Southborough, MA (US); Pat Koczela, Bridgewater, MA (US); Liam O'Shea, Westwood, MA (US); Andras Pungor, Bridgewater, MA (US); Leland Witherspoon, Elk Grove, CA (US); David Zitnick, Warwick, RI (US); R. Maxwell Flaherty, Auburndale, FL (US); J. Christopher Flaherty, Auburndale, FL (US)

(21) Appl. No.: 16/620,555

(22) PCT Filed: Jun. 11, 2018
(86) PCT No.: PCT/US18/36876

§ 371 Date: Dec. 9, 2019

Related U.S. Application Data
(60) Provisional application No. 62/517,433, filed on Jun. 9, 2017.

(51) Int. Cl. A61B 17/34; A61M 13/00

(52) U.S. Cl. CPC A61B 17/3423; A61B 2034/301; A61M 13/003

(57) ABSTRACT

A system for performing a medical procedure at a target location of a patient, including: an articulating probe including at least a distal portion; a feeder configured to advance, retract, and steer the articulating probe; a first introducer attached to the feeder and configured to slidingly receive the articulating probe; and a second introducer for providing access to an internal location of the patient. The first introducer operably attaches to the second introducer, such that the articulating probe can be advanced through the second introducer to the target location. The system further includes at least one sealing element configured to maintain insufflation pressure at the target location.

### RELATED APPLICATIONS

This application claims the benefit of U.S. Provisional Application No. 62/517,433, filed Jun. 9, 2017, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, the content of which is incorporated herein by reference in its entirety.

This application is related to U.S. patent application Ser. No. 14/892,750, filed Nov. 20, 2015, the content of which is incorporated herein by reference in its entirety.

This application is related to U.S. patent application Ser. No. 15/899,826, filed Feb. 20, 2018, the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, the content of which is incorporated herein by reference in its entirety.

This application is related to U.S. patent application Ser. No. 13/880,525, filed Apr. 19, 2013, now U.S. Pat. No. 8,992,421, the content of which is incorporated herein by reference in its entirety.

This application is related to U.S. patent application Ser. No. 14/587,166, filed Dec. 31, 2014, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US12/40414, filed Jun. 1, 2012, the content of which is incorporated herein by reference in its entirety.

This application is related to U.S. patent application Ser. No. 14/119,316, filed Nov. 21, 2013, the content of which is incorporated herein by reference in its entirety.

This application is related to U.S. Provisional Application Ser. No. 62/504,175, filed May 10, 2017, the content of which is incorporated herein by reference in its entirety.

This application is related to PCT Application No. PCT/US18/31774, filed May 9, 2018, the content of which is incorporated herein by reference in its entirety.

This application is related to U.S. Provisional Application Ser. No. 61/412,733, filed Nov. 11, 2010, the content of which is incorporated herein by reference in its entirety.

This application is related to PCT Application No PCT/US2011/060214, filed Nov. 10, 2011, the content of which is incorporated herein by reference in its entirety.

This application is related to U.S. patent application Ser. No. 13/884,407, filed May 9, 2013, now U.S. Pat. No. 9,649,163, the content of which is incorporated herein by reference in its entirety.

This application is related to U.S. patent application Ser. No. 15/587,832, filed May 5, 2017, the content of which is incorporated herein by reference in its entirety.

This application is related to U.S. Provisional Application Ser. No. 61/472,344, filed Apr. 6, 2011, the content of which is incorporated herein by reference in its entirety.

This application is related to PCT Application No. PCT/US12/32279, filed Apr. 5, 2012, the content of which is incorporated herein by reference in its entirety.

This application is related to U.S. patent application Ser. No. 14/008,775, filed Sep. 30, 2013, now U.S. Pat. No. 9,962,179, the content of which is incorporated herein by reference in its entirety.

This application is related to U.S. patent application Ser. No. 14/944,665, filed Nov. 18, 2015, the content of which is incorporated herein by reference in its entirety.

This application is related to U.S. patent application Ser. No. 14/945,685, filed Nov. 19, 2015, the content of which is incorporated herein by reference in its entirety.

This application is related to U.S. Provisional Application Ser. No. 61/534,032 filed Sep. 13, 2011, the content of which is incorporated herein by reference in its entirety.

This application is related to PCT Application No. PCT/US12/54802, filed Sep. 12, 2012, the content of which is incorporated herein by reference in its entirety.

This application is related to U.S. patent application Ser. No. 14/343,915, filed Mar. 10, 2014, now U.S. Pat. No. 9,757,856, issued Sep. 12, 2017, the content of which is incorporated herein by reference in its entirety.

This application is related to U.S. patent application Ser. No. 15/064,043, filed Mar. 8, 2016, now U.S. Pat. No. 9,572,628, issued Feb. 21, 2017, the content of which is incorporated herein by reference in its entirety.

This application is related to U.S. patent application Ser. No. 15/684,268, filed Aug. 23, 2017, the content of which is incorporated herein by reference in its entirety.

This application is related to U.S. Provisional Application Ser. No. 61/368,257, filed Jul. 28, 2010, the content of which is incorporated herein by reference in its entirety.

This application is related to PCT Application No PCT/US2011/044811, filed Jul. 21, 2011, the content of which is incorporated herein by reference in its entirety.

This application is related to U.S. patent application Ser. No. 13/812,324, filed Jan. 25, 2013, now U.S. Pat. No. 9,901,410, issued Feb. 27, 2018, the content of which is incorporated herein by reference in its entirety.

This application is related to U.S. patent application Ser. No. 15/874,189, filed Jan. 18, 2018, the content of which is incorporated herein by reference in its entirety.

This application is related to U.S. Provisional Application Ser. No. 61/578,582, filed Dec. 21, 2011, the content of which is incorporated herein by reference in its entirety.

This application is related to PCT Application No. PCT/US12/70924, filed Dec. 20, 2012, the content of which is incorporated herein by reference in its entirety.

This application is related to U.S. patent application Ser. No. 14/364,195, filed Jun. 10, 2014, now U.S. Pat. No. 9,364,955 issued Jun. 14, 2016, the content of which is incorporated herein by reference in its entirety.

This application is related to U.S. patent application Ser. No. 15/180,503, filed Jun. 13, 2016, now U.S. Pat. No. 9,821,477, issued Nov. 21, 2017, the content of which is incorporated herein by reference in its entirety.

This application is related to U.S. patent application Ser. No. 15/786,901, filed Oct. 18, 2017, the content of which is incorporated herein by reference in its entirety.

This application is related to U.S. Provisional Application Ser. No. 61/681,340, filed Aug. 9, 2012, the content of which is incorporated herein by reference in its entirety.

This application is related to PCT Application No. PCT/US13/54326, filed Aug. 9, 2013, the content of which is incorporated herein by reference in its entirety.

This application is related to U.S. patent application Ser. No. 14/418,993, filed Feb. 2, 2015, now U.S. Pat. No. 9,675,380 issued Jun. 13, 2017, the content of which is incorporated herein by reference in its entirety.

This application is related to U.S. patent application Ser. No. 15/619,875, filed Jun. 12, 2017, the content of which is incorporated herein by reference in its entirety.

This application is related to U.S. Provisional Application Ser. No. 61/751,498, filed Jan. 11, 2013, the content of which is incorporated herein by reference in its entirety.

This application is related to PCT Application No. PCT/US14/10808, filed Jan. 9, 2014, the content of which is incorporated herein by reference in its entirety.

This application is related to U.S. patent application Ser. No. 14/759,020, filed Jan. 9, 2014, the content of which is incorporated herein by reference in its entirety.

This application is related to U.S. Provisional Application Ser. No. 61/656,600, filed Jun. 7, 2012, the content of which is incorporated herein by reference in its entirety.

This application is related to PCT Application No. PCT/US13/43858, filed Jun. 3, 2013, the content of which is incorporated herein by reference in its entirety.

This application is related to U.S. patent application Ser. No. 14/402,224, filed Nov. 19, 2014, the content of which is incorporated herein by reference in its entirety.

This application is related to U.S. Provisional Application Ser. No. 61/825,297, filed May 20, 2013, the content of which is incorporated herein by reference in its entirety.

This application is related to PCT Application No. PCT/US13/38701, filed May 20, 2014, the content of which is incorporated herein by reference in its entirety.

This application is related to U.S. patent application Ser. No. 14/888,541, filed Nov. 2, 2015, now U.S. Pat. No. 9,517,059, issued Dec. 13, 2016, the content of which is incorporated herein by reference in its entirety.

This application is related to U.S. patent application Ser. No. 15/350,549, filed Nov. 14, 2016, the content of which is incorporated herein by reference in its entirety.

This application is related to U.S. Provisional Application Ser. No. 61/818,878, filed May 2, 2013, the content of which is incorporated herein by reference in its entirety.

This application is related to PCT Application No. PCT/US14/36571, filed May 2, 2014, the content of which is incorporated herein by reference in its entirety.

This application is related to U.S. patent application Ser. No. 14/888,189, filed Oct. 30, 2015, now U.S. Pat. No. 9,913,695, issued Mar. 13, 2018, the content of which is incorporated herein by reference in its entirety.

This application is related to U.S. patent application Ser. No. 14/916,664, filed Mar. 9, 2018, the content of which is incorporated herein by reference in its entirety.

This application is related to U.S. Provisional Application Ser. No. 61/909,605, filed Nov. 27, 2013, the content of which is incorporated herein by reference in its entirety.

This application is related to U.S. Provisional Application Ser. No. 62/052,736, filed Sep. 19, 2014, the content of which is incorporated herein by reference in its entirety.

This application is related to PCT Application No. PCT/US14/67091, filed Nov. 24, 2014, the content of which is incorporated herein by reference in its entirety.

This application is related to U.S. patent application Ser. No. 15/038,531, filed May 23, 2016, the content of which is incorporated herein by reference in its entirety.

This application is related to U.S. Provisional Application Ser. No. 62/008,453 filed Jun. 5, 2014, the content of which is incorporated herein by reference in its entirety.

This application is related to PCT Application No. PCT/US15/34424, filed Jun. 5, 2015, the content of which is incorporated herein by reference in its entirety.

This application is related to U.S. patent application Ser. No. 15/315,868, filed Dec. 2, 2016, the content of which is incorporated herein by reference in its entirety.

This application is related to U.S. Provisional Application Ser. No. 62/150,223, filed Apr. 20, 2015, the content of which is incorporated herein by reference in its entirety.

This application is related to U.S. Provisional Application Ser. No. 62/299,249, filed Feb. 24, 2016, the content of which is incorporated herein by reference in its entirety.

This application is related to PCT Application No. PCT/US16/28374, filed Apr. 20, 2016, the content of which is incorporated herein by reference in its entirety.

This application is related to U.S. patent application Ser. No. 15/567,109, filed Oct. 17, 2017, the content of which is incorporated herein by reference in its entirety.

This application is related to U.S. Provisional Application Ser. No. 62/401,390, filed Sep. 29, 2016, the content of which is incorporated herein by reference in its entirety.

This application is related to PCT Application No. PCT/US17/54297, filed Sep. 29, 2017, the content of which is incorporated herein by reference in its entirety.

This application is related to U.S. Provisional Application Ser. No. 62/481,309, filed Apr. 4, 2017, the content of which is incorporated herein by reference in its entirety.

This application is related to U.S. Provisional Application Ser. No. 62/598,812, filed Dec. 14, 2017, the content of which is incorporated herein by reference in its entirety.

This application is related to U.S. Provisional Application Ser. No. 62/617,513, filed Jan. 15, 2018, the content of which is incorporated herein by reference in its entirety.

This application is related to PCT Application No. PCT/US18/026016, filed Apr. 4, 2018, the content of which is incorporated herein by reference in its entirety.

This application is related to U.S. Provisional Application Ser. No. 62/533,644, filed Jul. 17, 2017, the content of which is incorporated herein by reference in its entirety.

This application is related to U.S. Provisional Application Ser. No. 62/614,263, filed Jan. 5, 2018, the content of which is incorporated herein by reference in its entirety.

This application is related to U.S. Provisional Application Ser. No. 62/582,283, filed Nov. 6, 2017, the content of which is incorporated herein by reference in its entirety.

This application is related to U.S. Provisional Application Ser. No. 62/614,346, filed Jan. 5, 2018, the content of which is incorporated herein by reference in its entirety.

This application is related to U.S. Provisional Application Ser. No. 62/613,899, filed Jan. 5, 2018, the content of which is incorporated herein by reference in its entirety.

This application is related to U.S. Provisional Application Ser. No. 62/614,223, filed Jan. 5, 2018, the content of which is incorporated herein by reference in its entirety.

This application is related to U.S. Design application Ser. No. 29/632,148, filed Jan. 5, 2018, the content of which is incorporated herein by reference in its entirety.

This application is related to U.S. Provisional Application Ser. No. 62/614,224, filed Jan. 5, 2018, the content of which is incorporated herein by reference in its entirety.

This application is related to U.S. Provisional Application Ser. No. 62/614,228, filed Jan. 5, 2018, the content of which is incorporated herein by reference in its entirety.

This application is related to U.S. Provisional Application Ser. No. 62/614,225, filed Jan. 5, 2018, the content of which is incorporated herein by reference in its entirety.

This application is related to U.S. Provisional Application Ser. No. 62/614,240, filed Jan. 5, 2018, the content of which is incorporated herein by reference in its entirety.

This application is related to U.S. Provisional Application Ser. No. 62/614,235, filed Jan. 5, 2018, the content of which is incorporated herein by reference in its entirety.

This application is related to U.S. Pat. No. 9,011,318, issued Apr. 21, 2015, the content of which is incorporated herein by reference in its entirety.

### FIELD

The present inventive concepts relate generally to surgical access systems and tools for performing surgical procedures. In particular, systems that provide for an insufflated target area for performing a surgical procedure are described.

### BACKGROUND

As less invasive medical techniques and procedures become more widespread, medical professionals such as surgeons may require articulating surgical tools, such as endoscopes, to perform such less invasive medical techniques and procedures that require access to locations within the patient, such as a site accessible through the mouth or other natural orifice, or a site accessible through an incision through the patient's skin. There is a need for flexible robotic instrumentation that allows for minimally invasive access and visualization of surgical procedures in any insufflated area not easily accessible through straight line of sight surgical instrumentation.

### SUMMARY

Embodiments of the systems, devices and methods described herein can be directed to systems, devices and methods for performing medical techniques and procedures that require access to locations within the patient, such as a site accessible through the mouth or other natural orifice, or a site accessible through an incision through the patient's skin.

According to an aspect of the present inventive concepts, a system for performing a medical procedure at a target location of a patient comprises: an articulating probe comprising at least a distal portion; a feeder configured to advance, retract, and steer the articulating probe; a first introducer attached to the feeder and configured to slidingly receive the articulating probe; and a second introducer for providing access to an internal location of the patient. The first introducer operably attaches to the second introducer, such that the articulating probe can be advanced through the first and second introducers to the target location. The system further comprises at least one sealing element configured to maintain insufflation pressure at the target location.

In some embodiments, the articulating probe can comprise an inner probe comprising multiple articulating inner links and an outer probe surrounding the inner probe and comprising multiple articulating outer links. The one of the inner probe or the outer probe can be configured to transition between a rigid mode and a flexible mode, and the other of the inner probe or the outer probe can be configured to transition between a rigid mode and a flexible mode and to be steered. The inner probe can comprise an elongated proximal link. The elongated proximal link can remain within a straight portion of the feeder. The system can further comprise a base unit including a carriage assembly, and the elongated proximal link can comprise a projection. The outer probe can comprise an elongated proximal link. The elongated proximal link can remain within a straight portion of the feeder. The system can further comprise a base unit including a carriage assembly, and the elongated proximal link can comprise a projection. The outer probe can comprise a plurality of proximal links and a plurality of distal links. The distal links can be dissimilar to the proximal links. The distal links can comprise a shorter length than the proximal links. The distal links can be constructed and arranged to accommodate a smaller turning radius than the proximal links. The outer probe can comprise a distal link assembly. The system can further comprise a camera assembly including a camera cable, and the camera cable can removably attach to the distal link assembly. The articulating probe can comprise one or more internal working channels. Each of the inner and outer links can comprise one or more recesses such that the one or more recesses can comprise the one or more internal working channels. The one or more internal working channels can slidingly receive an elongate element. The elongate element can comprise a tube with a lumen therethrough. The tube can be configured to at least one of provide or remove one or more fluids to the target location, such as to provide at least one of insufflation or desufflation. The tube can be configured to provide an irrigation fluid to flush at least a portion of the target location. The elongate element can comprise a tool. The one or more internal working channels can comprise the at least one sealing element. The at least one sealing element can be positioned at a proximal end of the one or more internal working channels. The at least one sealing element can be positioned at a distal end of the one or more working channels. The at least one sealing element can be configured to limit an ingress of a fluid from the outside of the articulating probe into the one or more internal working channels.

In some embodiments, the first introducer can surround a first chamber. The first chamber can be configured to maintain a pressure within the first chamber. The first introducer can comprise an opening into the first chamber, and the opening can be configured to slidingly receive the articulating probe. The at least one sealing element can comprise a sealing element located between the opening and the articulating probe. The at least one sealing element can be configured to limit an egress of a fluid from the first chamber between the opening and the articulating probe. The articulating probe can comprise a sheath. The least one sealing element can be configured to provide a seal between the opening and the sheath. The at least one sealing element can be configured to limit an ingress and/or egress of a fluid from the first chamber to between the opening and the sheath. The system can further comprise a camera assembly including a camera cable and the first introducer can comprise an opening into the first chamber, and the opening can be configured to slidingly receive the camera cable. The opening can comprise the at least one sealing element, and the at least one sealing element can be configured to maintain a seal between the opening and the camera cable. The at least one sealing element can be configured to limit an egress of a fluid from the first chamber between the opening and the camera cable. The system can further comprise at least one tool guide and the first introducer can comprise at least one access port into the first chamber, and the system can further comprise a connector configured to operably attach the at least one access port to the at least one tool guide. The at least one sealing element can comprise a sealing element located between the connector and the at least one access port. The at least one access port can comprise a compression ring configured to maintain a seal between the at least one access port and the connector. The compression ring can be configured to limit an egress of a fluid from the first chamber between the connector and the at least one access port. The at least one sealing element can comprise a sealing element located between the connector and the at least one tool guide. The connector can comprise at least one projection and the tool guide can comprise at least one recess, and the at least one projection can be configured to engage the at least one recess so as to maintain a seal between the at least one projection and the at least one recess. The connector can comprise an elastic material configured to maintain a seal between the connector and the at least once tool guide. The at least one sealing element can be configured to limit an egress of a fluid from the first chamber between the connector and the at least one tool guide. The connector can comprise at least one flexible portion. The second introducer can surround a second chamber, and the second chamber can be configured to operably attach to the first chamber of the first introducer. The at least one sealing element can comprise a sealing element located between the first chamber and the second chamber. The at least one sealing element can be configured to limit an egress of a fluid from at least one of the first and second chamber between the first and second chambers. The first introducer can comprise a cover and the first chamber can comprise an opening, and the cover can be constructed and arranged to surround the opening of the first chamber. The at least one sealing element can comprise a sealing element located between the cover and the first introducer. The at least one sealing element can comprise a gasket positioned proximate the perimeter of the opening. The at least one sealing element can be configured to limit an egress of a fluid from the first chamber between the cover and the first introducer. The cover can comprise at least one retention clip configured to releasably secure the cover to the first introducer. The first introducer can comprise at least one tool port providing access into the first chamber. The at least one tool port can comprise at least one sealing element, and the at least one sealing element can comprise a valve. The at least one sealing element can be configured to slidingly receive an elongate device. The at least one sealing element can be configured to limit an egress of a fluid from the first chamber via the at least one tool port.

In some embodiments, the first introducer can be removably attached to the feeder.

In some embodiments, the first introducer can comprise a connector comprising an upper portion and a lower portion. The connector can comprise a bellows configured to flexibly connect the upper portion to the lower portion, and the bellows can comprise a first end sealed to the upper portion and a second end sealed to the lower portion. The lower portion can be configured to operably attach to the second introducer.

In some embodiments, the first introducer can comprise a housing comprising multiple discrete components. The housing can be constructed and arranged to surround at least a portion of the articulating probe.

In some embodiments, the second introducer can be constructed and arranged to be inserted into a natural orifice of the patient. The natural orifice can comprise the anus. The natural orifice can comprise an anatomical location selected from the group consisting of: anus; vagina; meatus; nostril; mouth; ear; and combinations thereof. The at least one sealing element can comprise a sealing element located between the second introducer and the natural orifice. The at least one sealing element can be configured to limit an egress of a fluid between the second introducer and the natural orifice. The second introducer can comprise a rectoscope.

In some embodiments, the second introducer can be constructed and arranged to be inserted through an incision into the patient. The second introducer can be constructed and arranged to be inserted through an incision into the abdomen of the patient. The at least one sealing element can comprise a sealing element located between the second introducer and the incision. The at least one sealing element can be configured to limit an egress of fluid between the second introducer and the incision.

In some embodiments, the second introducer can comprise an insufflation port assembly configured to provide an insufflation fluid. The insufflation port assembly can comprise a lumen, and the lumen can terminate proximate a distal end of the second introducer.

In some embodiments, the second introducer can comprise a distal end and the articulating probe can comprise a maximum width, and the distal end can be constructed and arranged to slidingly receive the maximum width of the articulating probe.

In some embodiments, the at least one sealing element can be configured to limit a leakage of an insufflation fluid from the target location. The at least one sealing element can be configured to limit the leakage of the insufflation fluid to a maximum leakage rate selected from the group consisting of: 5 L/min; 4 L/min; 3 L/min; 2L/min; and combinations thereof. The at least one sealing element can be configured to limit the leakage of the insufflation fluid to a maximum leakage rate selected from the group consisting of: 5 L/min; 4 L/min; 3 L/min; 2 L/min; and combinations thereof. The at least one sealing element can be configured to slidingly surround an elongate element, and the at least one sealing element can be further configured to maintain a seal without surrounding the elongate element.

In some embodiments, the at least one sealing element can comprise a space between a lumen and an elongate element, and the lumen can be configured to slidingly receive the elongate element, and a resistance between the lumen and the elongate element maintains a seal between the two.

In some embodiments, the at least one sealing element can comprise a sheath configured to surround at least a distal portion of the articulating probe. The sheath can be configured to limit an egress of a fluid from the outside to the inside of the articulating probe. The sheath can be configured to limit an egress of a fluid from the outside to the inside of the articulating probe. The sheath can comprise an elastic material. The sheath can comprise a durometer of less than 90 A, less than 85 A, and/or less than 80 A. The articulating probe can comprise at least one recess, and the sheath can be secured to the articulating probe within the at least one recess. The sheath can be adhesively attached within the at least one recess. The sheath can be adhesively attached to at least a portion of the articulating probe. A proximal end of the sheath can be adhesively attached to at least a portion of the articulating probe. The system can be configured to advance the probe to a maximum extension, and the sheath can comprise a proximal end, and the proximal end of the sheath can be positioned within the feeder when the probe is advanced to the maximum extension. The probe can further comprise a distal link and a distal link assembly with a proximal wall, and the sheath can be sealed to the distal link, and the distal link can be sealed to the distal link assembly proximate the proximal wall.

In some embodiments, the system can further comprise a base unit, and the feeder can be removably attached to the base unit. The base unit can comprise an emergency release mechanism. The emergency release mechanism can comprise one or more buttons. The emergency release mechanism can be configured to disconnect the feeder from the base unit

In some embodiments, the target location can comprise an anatomical location selected from the group consisting of: colon; intestine; esophagus; nasal passageway; vaginal canal; ear canal; and combinations thereof.

In some embodiments, the system can further comprise a console. The console can comprise a user interface, and the user interface can be configured to receive one or more commands from a user. The user interface can comprise at least one user input device, and the at least one user input device can comprise at least one of a joystick, multi-axis input device, mouse, keyboard, touchscreen device, and combinations thereof. The user interface can comprise at least one output device, and the at least one output device can comprise at least one of a monitor, speaker, haptic controller, indicator light, and combinations thereof. The console can comprise at least one functional element. The at least one functional element can comprise at least one of a sensor or transducer. The at least one functional element can comprise a sensor configured to produce a signal related to a proper operation of the console by a user. The at least one functional element can comprise at least one of an accelerometer and gyroscope.

In some embodiments, the system can further comprise a base unit. The base unit can comprise one or more algorithms. The base unit can comprise an image processing unit. The base unit can comprise one or more alignment pins and the feeder can comprise one or more alignment holes, and the one or more alignment holes can be constructed and arranged to slidingly receive the one or more alignment pins. The base unit can comprise a first electrical connector and the feeder can comprise a second electrical connector, and one of the alignment pins can be near the first electrical connector and one of the alignment holes can be near the second electrical connector. The one or more alignment pins can be constructed and arranged along a length of the base unit, and the one or more alignment holes can be constructed and arranged along a length of the feeder. The base unit can comprise a top assembly and a bottom assembly. The top assembly can be resposable. The top assembly can comprise a proximal portion and distal portion, and the distal portion can be constructed and arranged to extend towards the patient. The base unit can be configured to communicate with the feeder to control the articulating probe. The base unit can comprise one or more carriage assemblies configured to advance the articulating probe. The base unit can comprise one or more drive mechanisms configured to steer the articulating probe.

In some embodiments, the system can further comprise at least one tool guide. The at least one tool guide can comprise a sealing element comprising a valve. The at least one sealing element can be configured to limit an egress of a fluid from the at least one tool guide from the valve. The at least one sealing element can be configured to slidingly receive an elongate device. The at least one sealing element can be configured to limit an egress of a fluid from the at least one tool guide between the valve and the elongate device. The elongate device can comprise a tool. The valve can be removably attached to a proximal end of the at least one tool guide. The at least one tool guide can comprise a tool guide extension. The system can further comprise at least one guide tube that can be configured to be slidingly received by the at least one tool guide, and the at least one guide tube can be operably attached to the distal portion of the articulating probe. The system can comprise two or more tool guides. The system can further comprise a connector configured to maintain a relative position between the two or more tool guides. The connector can comprise at least one attachment point configured to attach to a brace.

In some embodiments, the system can further comprise at least one tool. The at least one tool can comprise a tool with a flexible portion. The at least one tool can comprise a tool with a rigid portion. The at least one tool can comprise a tool selected from the group consisting of: a laser delivery element; scissor; blade; cautery element; suction element; irrigation element; grasper; surgical stapler; tissue securing element; and combinations thereof.

In some embodiments, the system can further comprise camera assembly comprising a camera, a cable, and a connector. The camera assembly can be reusable. The cable can be positioned along at least a portion of the articulating probe. The articulating probe can comprise a distal link assembly, and the camera can be configured to operably attach to the distal link assembly.

In some embodiments, the system can further comprise at least one functional element. The first introducer can comprise a chamber, and the at least one functional element can be positioned within the chamber. The second introducer can comprise a chamber, and the at least one functional element can be positioned within the chamber. The feeder can comprise a chamber, and the at least one functional element can be positioned within the chamber. The articulating probe can comprise a chamber, and the at least one functional element can be positioned within the chamber. The at least one functional element can be configured to produce a signal related to at least one of a status of an insufflation or a status of a tissue treatment. The at least one functional element can comprise a sensor, and the sensor can comprise at least one of a pressure sensor, light sensor, position transducer, accelerometer; flow sensor; and combinations thereof

In some embodiments, the system can further comprise a cable management assembly comprising one or more cables configured to translate with the articulating probe. The cable management assembly can comprise a clip strip comprising a plurality of clips. The clip strip can be removably attached to the distal portion of the articulating probe. The clip strip can axially receive the one or more cables. The clip strip can be configured to limit slack in the one or more cables as the articulating probe retracts. The cable management assembly can comprise a clip positioned on a proximal portion of the articulating probe. The cable management assembly can comprise a ramp with a first end proximate the articulating probe and a second end radially offset from the articulating probe, and the one or more cables can be positioned on the ramp from the first end to the second end. The ramp can comprise one or more radially offset seals, and the one or more cables can translate through at least one of the radially offset seals.

In some embodiments, the second introducer comprises a connector portion, a flexible portion, and a rigid portion that couples the connector portion to the flexible portion.

In some embodiments, the second introducer is constructed and arranged to allow access into the patient's body by applying a conformable outward force to achieve a desired dilation at the insertion location.

In some embodiments, the connector portion comprises an opening constructed and arranged to receive the articulating probe.

In some embodiments, the connector portion is constructed and arranged to couple with a bellows that provides a flexible connection between the connector portion and a connector of the first introducer.

In some embodiments, the flexible portion comprises an access conduit and an anchoring flange.

In some embodiments, the access conduit comprises a width to receive a distal link assembly of the articulating probe.

In some embodiments, the access conduit applies an outward radial force on the patient's body when inserted.

In some embodiments, the access conduit applies an outward radial force on the patient's sphincter when inserted.

In some embodiments, the anchoring flange is positioned at a distal end of the flexible portion and is constructed and arranged to maintain the placement of the second introducer within the patient's body.

In some embodiments, at least one of the anchoring flange, access conduit, or flexible portion comprises a circular cross-section.

In some embodiments, at least one of the anchoring flange, access conduit, or flexible portion comprises an oval or elliptical cross-section.

In some embodiments, a length of the access conduit region is constructed and arranged to accommodate one or more patient parameters.

In some embodiments, the flexible portion provides a resistance to sphincter contraction and allows for collapse during insertion into the patient.

In some embodiments, the flexible portion is sealed to the connector portion to prevent fluid release during an insufflation procedure.

In some embodiments, the rigid portion is directly connected to the flexible portion.

In some embodiments, the second introducer includes one or more insufflation ports.

In some embodiments, the connector portion includes a support arm constructed and arranged to removably attach the second introducer to a stabilizing brace.

In some embodiments, the flexible portion is constructed and arranged to collapse for manual insertion into the patient's body at a reduced width.

In some embodiments, the second introducer comprises a rigid introducer portion and a flexible portion, each interchangeably attachable to a rigid portion and a bellows.

In some embodiments, the second introducer comprises a connector portion, an access conduit, and an adapter coupled to the distal end of the second introducer.

In some embodiments, the access conduit comprises a width that is sufficient to receive a distal link assembly of the articulating probe.

In some embodiments, the adapter comprises a conformable sleeve, an anchoring ring coupled to the distal end of the conformable sleeve, a locking collar positioned around the conformable sleeve, a roller lock, a spring, and a roller coupled to the spring.

In some embodiments, the sleeve is constructed and arranged to allow the orientation of the anchoring ring to be adjusted.

In some embodiments, the anchoring ring is constructed and arranged to be positioned at the interior wall of the patient's sphincter.

In some embodiments, the sleeve is constructed and arranged to be pulled in the proximal direction and passes between the roller and the locking collar.

In some embodiments, the sleeve is positioned between the roller and the locking collar.

In some embodiments, the spring plate imparts a biasing force to push the roller against the sleeve, pinching the sleeve against the locking collar.

In some embodiments, the tension on the sleeve between the anchoring ring and the rollers is adjustable.

In some embodiments, the roller is constructed and arranged to operate as a ratchet.

In some embodiments, the sleeve is constructed and arranged to dilate so that its diameter can be reduced and expanded.

In some embodiments, the sleeve is constructed and arranged to dilate so that its width can be reduced and expanded.

In some embodiments, the path of the roller comprises a surface comprising an acute angle to the corresponding surface on the locking ring, which pinches the sleeve.

In some embodiments, the adapter is constructed and arranged to allow access into the patient's body by applying a conformable outward force to achieve a desired dilation.

In some embodiments, the adapter is configured to seal against the second introducer.

In some embodiments, the second introducer comprises a connector portion, an access conduit, and a projection.

In some embodiments, the access conduit comprises a width that is sufficient to receive a distal link assembly of the articulating probe.

In some embodiments, the system further comprises an adapter coupled to a distal end of the second introducer.

In some embodiments, the adapter comprises: a proximal end comprising a proximal flange and a handle; a distal end comprising an anchoring flange; an intermediate region positioned between the proximal end and the distal end, the intermediate region comprising a placement feature; and an access conduit that extends through the proximal end, the distal end, and the intermediate region.

In some embodiments, the anchoring flange maintains the placement of the introducer within the patient.

In some embodiments, the adapter is flexible to allow for easier manual placement into the patient's body.

In some embodiments, the second introducer is constructed and arranged to be inserted into the adapter.

In some embodiments, the adapter is constructed and arranged to dilate to a desired diameter.

In some embodiments, the adapter comprises one or more placement features.

In some embodiments, the placement features are constructed and arranged to mate with a projection of the second introducer to facilitate placement of the second introducer.

In some embodiments, the second introducer includes a support constructed and arranged to removably attach the second introducer to a stabilizing brace.

According to another aspect of the present inventive concepts, a shaped instrument support comprises a shaped brace, coupled to one or more non-linear tool guides.

In some embodiments, the shaped brace is coupled between proximal ends of the one or more non-linear tool guides.

In some embodiments, the shaped brace is constructed and arranged to maintain a relative position between at least two of the non-linear tool guides.

In some embodiments, the shaped brace comprises a curved geometry in a plane transverse to an axis of extension of a first tool guide and an axis of extension of a second tool guide.

In some embodiments, the curved geometry in the shaped brace creates a viewing space region between the first and second tool guides.

In some embodiments, the first tool guide and second tool guide include proximal portions extending at a non-zero angle with respect to each other and wherein the first tool guide and second tool guide include distal portions extending in a direction parallel to each other.

In some embodiments, the one or more non-linear tool guides are configured to connect to a port.

In some embodiments, a distal portion of the one or more non-linear tool guides comprises a bend region.

In some embodiments, the bend region is constructed and arranged such that the distal portion extends along an axis orthogonal to a port.

In some embodiments, the one or more non-linear tool guides comprise a sealing segment positioned between a radial projection, a proximal stop, and a bend region.

In some embodiments, the one or more non-linear tool guides comprises a segment configured to slidingly seal to a projection of a sealing element.

In some embodiments, the segment is configured such that the projection slidingly engages the segment along its length, such that the one or more non-linear tool guides operably attach to an introducer in various positions.

In some embodiments, at least one of the one or more non-linear tool guides comprises a proximal stop configured as a physical limiter and/or visual marker to inform the user of the limits of the positioning.

In some embodiments, at least one of the non-linear tool guides comprises a bend region configured as a physical limiter and/or visual marker to inform the user of the limits of the positioning.

In some embodiments, a distal portion of at least one of the one or more non-linear tool guides extends orthogonally through openings of a port.

In some embodiments, the shaped instrument support further comprises one or more attachment points constructed and arranged to removably attach the instrument support to a stabilizing brace.

In some embodiments, the shaped brace comprises at least one threaded projection aligned with the position of each of the one or more non-linear tool guides.

In some embodiments, the shaped brace comprises two threaded projections such that each threaded projection is aligned with a corresponding tool guide.

In some embodiments, the at least one threaded projection comprises threads constructed and arranged to mate with a threaded adapter.

In some embodiments, the threaded adapter is configured to extend the length of the tool guide.

In some embodiments, the threaded adapter comprises threads constructed and arranged to mate with the threads of the corresponding threaded projection.

In some embodiments, the threaded adapter comprises a seal assembly constructed and arranged to maintain a seal.

In some embodiments, the threaded adapter comprises an 0-ring to prevent gas, or other fluid, from passing through the connection between the threads of the threaded adapter and the threads of the threaded projection.

According to another aspect of the present inventive concepts, a method of operating an articulating probe, and the articulating probe is advanced through a luminal pathway, the method comprises: introducing the articulating probe proximate a proximal end of a luminal pathway, the articulating probe comprises an inner and an outer probe and the inner and outer probes each include a plurality of links with a distal-most link; advancing both the inner and outer probes of the articulating probe in a double limp mode, and the inner and outer probe simultaneously advance each in a limp state within the luminal pathway; transitioning the inner probe from the limp state to a rigid state; continue advancing the articulating probe in a follow the leader mode, comprising: advancing the limp outer probe such that its distal-most link extends at least one link's length beyond the distal-most link of the inner probe, and the rigid inner probe is not advanced; transitioning the outer probe from the limp state to a rigid state and the inner probe from the rigid state to the limp state; advancing the limp inner probe such that its distal-most link is coextensive with the distal-most link of the outer probe, and the rigid outer probe is not advanced; the inner and outer probes are alternatively advanced to approach at least one target location within the luminal pathway, and the inner and/or outer probes are steerable in the limp state; performing at least one procedure at the at least one target location; and retracting the articulating probe from the luminal pathway such that both the inner and outer probes are retracted in the double limp mode and/or the follow the leader mode. The limp outer probe can be advanced such that its distal-most link extends between two and twenty link's length beyond the distal-most link of the inner probe.

According to another aspect of the present inventive concepts, a system for performing a medical procedure at a target location of a patient, comprises: an articulating probe comprising at least a distal portion; a feeder configured to advance, retract, and steer the articulating probe: the articulating probe comprises an inner probe comprising multiple articulating inner links and an outer probe surrounding the inner probe and comprising multiple articulating outer links; the outer probe comprises a plurality of proximal links and a plurality of distal links; the distal links comprise a shorter length than the proximal links; and the distal links are constructed and arranged to accommodate a smaller turning radius than the proximal links.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a schematic view of an articulating probe system, consistent with the present inventive concepts.

FIG. 2 illustrates an exploded perspective view of an articulating probe system, consistent with the present inventive concepts.

FIG. 3 illustrates perspective views of an articulating probe and a camera assembly for an articulating probe system, consistent with the present inventive concepts.

FIG. 4 illustrates a perspective view of the distal end of an articulating probe for an articulating probe system, consistent with the present inventive concepts.

FIG. 5 illustrates a cutaway perspective view of an introducer for an articulating probe system, consistent with the present inventive concepts.

FIG. 6 illustrates an exploded perspective view of an introducer for an articulating probe system, consistent with the present inventive concepts.

FIG. 7 illustrates a perspective view of a flexible connection between an introducer connector portion and a body introducer for an articulating probe system, consistent with the present inventive concepts.

FIGS. 8 and 8A illustrate unassembled and assembled views, respectively of a tool, an instrument support, and an introducer of an articulating probe system, consistent with the present inventive concepts.

FIGS. 9A-C are graphic demonstrations of an articulating probe, consistent with the present inventive concepts.

FIG. 10 is a flow chart of a method for advancing and/or retracting an articulating probe during a procedure, consistent with the present inventive concepts.

FIG. 11 illustrates a cross-sectional view of an articulating probe comprising inner and outer links, consistent with the present inventive concepts.

FIG. 12 illustrates cross sectional and side views of proximal and distal outer links of an articulating probe, consistent with the present inventive concepts.

FIG. 13 illustrates a sectional side view of a body introducer, consistent with the present inventive concepts.

FIG. 14 illustrates a sectional side view of a body introducer, consistent with the present inventive concepts.

FIGS. 15A and 15B illustrates a sectional side view and a perspective view, respectively of a body introducer and an adapter consistent with the present inventive concepts.

FIG. 16A illustrates a perspective view of a shaped instrument support, consistent with the present inventive concepts.

FIG. 16B illustrates a top view of a shaped instrument support, consistent with the present inventive concepts.

FIG. 16C illustrates a side view of a shaped instrument support, consistent with the present inventive concepts.

FIG. 17 illustrates a sectional side view of a shaped instrument support, consistent with the present inventive concepts.

### DETAILED DESCRIPTION OF THE DRAWINGS

Reference will now be made in detail to the present embodiments of the technology, examples of which are illustrated in the accompanying drawings. The same reference numbers are used throughout the drawings to refer to the same or like parts.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate” shall include locations relatively close to, on, in and/or within a referenced component or other location.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

The term “working channel” where used herein is to be taken to include a lumen or other elongate space through which an elongate component such as an elongate tool, a guide tube or other tube, or other elongate element can be translated.

The term “seal”, and its iterations, where used herein is to be taken to describe an arrangement that prevents or at least limits leakage of a fluid (e.g. air or water) out of a space (e.g. an internal body space of a patient and/or a chamber of a device). A seal can describe a fluid-tight seal or a seal that simply limits egress of fluid. A seal can be provided by one or more sealing elements, such as an adhesive, 0-ring, gasket, and/or washer that provides a seal between two or more components, or between a component and tissue of a patient (e.g. tissue of an orifice of a patient). A sealed environment (also referred to as simply a “seal”) can be provided to allow one or more internal body locations of a patient to be insufflated, such as to distend tissue to enhance access to a target site within the patient. The seal can be configured to limit leakage of fluid from the sealed environment, such as to limit the number of times insufflation fluid needs to be delivered into the environment to maintain sufficient distension of the tissue. In some embodiments, a sufficient seal comprises a seal that limits leakage to a rate of no more than 5 L/min, no more than 4 L/min, no more than 3 L/min, and/or no more than 2 L/min (e.g. at an insufflation pressure between 0.2 psi and 0.4 psi, or approximately 0.3 psi).

The term “pressurize”, and its iterations, where used herein is to be taken to describe a process of providing a fluid to a defined volume, the volume constructed and arranged to maintain the fluid for at least a period of time such that a measurable pressure change occurs within the volume while and/or after the fluid is provided (i.e. the volume is capable of maintaining fluid pressure). For example, a “pressurizable” volume can comprise a volume within a component, having all openings from the volume sealed to limit fluid egress, and/or operably attached to one or more additional pressurizable volumes, the multiple pressurizable volumes defining a closed system, capable of maintaining a pressure.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the disclosure concepts have been simplified to focus on elements that are relevant for a clear understanding of the inventive concepts, while eliminating, for purposes of clarity, other elements that those of ordinary skill in the art will appreciate may also comprise a portion of the inventive conceptse. However, because such elements are well known in the art, and because they do not necessarily facilitate a better understanding of the inventive concepts , a description of such elements is not provided herein.

Provided herein are systems for providing access to a target site within a patient. The system can include one or more tools for performing a surgical, diagnostic, and/or other clinical procedure (“surgical procedure” herein) on the patient. Each of the components of the system of the present inventive concepts can comprise one or more sealing elements, such as to support an insufflation procedure (e.g. prevent or at least limit leaks of insufflation fluid from an insufflated area within the patient).

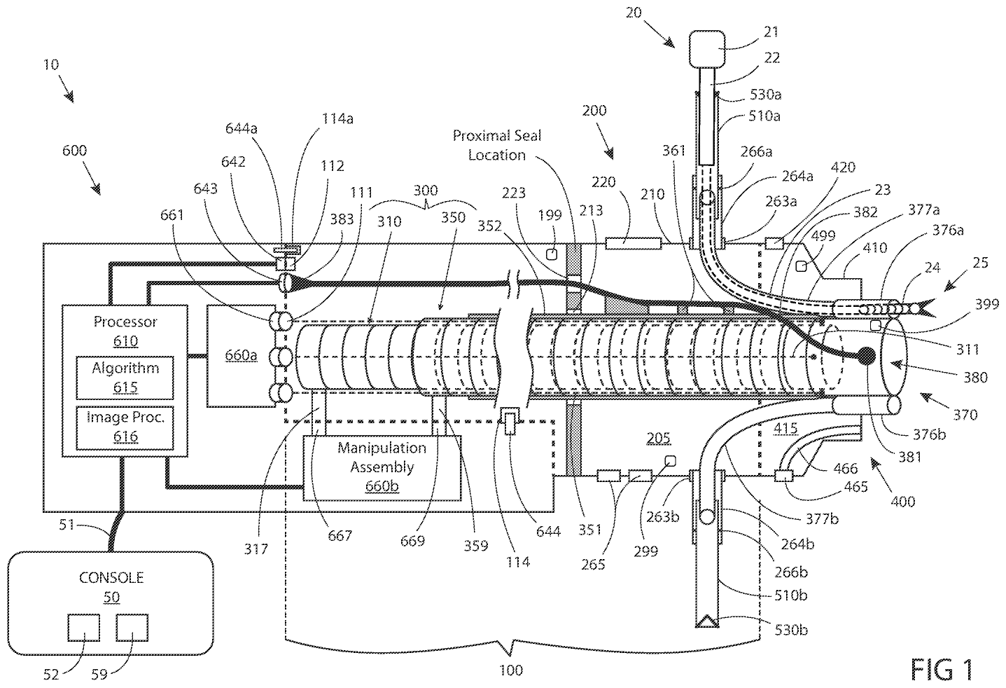

Referring now to FIG. 1, a schematic view of an articulating probe system is illustrated, consistent with the present inventive concepts. Articulating probe system 10 includes a base unit 600 configured to operably attach to a console 50 and a feeder 100 comprising an articulating probe 300. Feeder 100 further comprises a first introducer, introducer 200, configured to operably and fluidly attach (e.g. an attachment that maintains a fluid-tight seal or other seal) to a second introducer, body introducer 400. Body introducer 400 can be configured to provide surgical access to a body cavity (e.g. a natural body cavity such as the colon or rectum) within which cavity is a target location (e.g. a tissue or target location for a surgical procedure to be performed, such as one or more locations within: the colon; the intestine; the esophagus; a nasal passageway; the vaginal canal; an ear canal; an artificially insufflated cavity in the abdomen; and/or other body lumen or body location). For example, body introducer 400 can comprise a rectoscope configured to provide sealed colorectal access, such that insufflation of the rectum and/or colon can be performed and maintained (e.g. maintained by supplying minimal insufflation fluid throughout the procedure).

Articulating probe system 10 can be used to perform a surgical procedure at a target location within a patient's body cavity (e.g. a non-line of site surgical procedure via a natural orifice such as the anus or a percutaneously accessed location such as the abdomen), and provide sealed access into the orifice such that the cavity can be insufflated (e.g. by a component of system 10), without unwanted loss of significant insufflation through the access point or any component of system 10. In some embodiments, body introducer 400 provides sealed access to a location via an orifice selected from the group consisting of: the anus; the vagina; the meatus (distal end of the urethra); a nostril; the mouth; an ear; a surgical incision; and combinations of one or more of these.

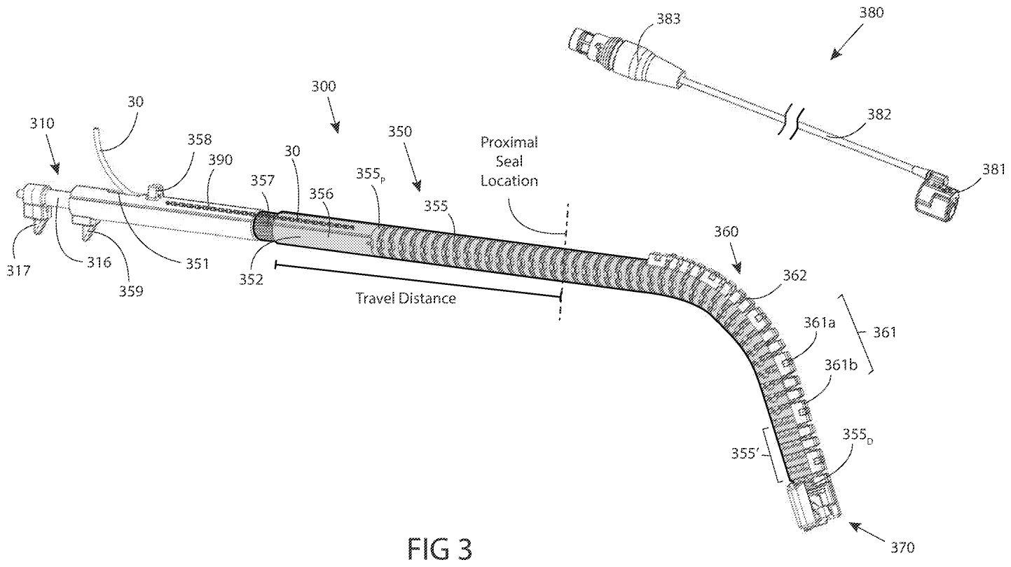

Articulating probe 300 can comprise a multi-link probe including an inner probe comprising a set of inner links, and a surrounding outer probe comprising a set of outer links, such as is described in reference to applicant's co-pending U.S. patent application Ser. No. 15/378,723, filed Dec. 14, 2016, the contents of which are incorporated herein by reference in their entirety for all purposes, and/or as described in further detail herebelow in reference to FIGS. 3, 4, and 9A-C. In some embodiments, articulating probe 300 comprises an inner probe 310 and an outer probe 350, such that outer probe 350 slidingly receives inner probe 310. Outer probe 350 can comprise one or more steering cables, cables 351, configured to steer outer probe 350, and/or transition outer probe 350 between limp and rigid states, as described herein. Inner probe 310 can comprise one or more steering cables, cable 311, also configured to steer inner probe 310 and/or transition inner probe 310 between limp and rigid states as described here. In some embodiments, only one of outer probe 350 or inner probe 310 can be steered, while both can transition between limp and rigid states. Probe 300 can comprise an outer sheath, sheath 352 shown, configured to limit fluid ingress (or egress) from outside probe 300 to inside probe 300. For example, as described herein, when probe 300 is advanced to a surgical site within an insufflated environment, sheath 352 provides a seal around probe 300, limiting insufflation gases from entering probe 300. Sheath 352 can comprise a soft and/or flexible material (e.g. to reduce undesired stiffening of probe 300). In some embodiments, sheath 352 comprises an elastomer material, such as Pellethane® or other thermoplastic polyurethane, or similar.

Console 50 can comprise a user interface 52 configured to receive commands from a surgeon, technician, and/or other operator of articulating probe system 10. User interface 52 can include one or more user input devices, such as a joystick, a multi axis input device, a mouse, a keyboard, and/or a touchscreen device. User interface 52 can further include one or more output devices, such as a monitor, speaker, haptic controller, and/or an indicator light. In some embodiments, console 50 includes one or more functional elements 59, such as a sensor or a transducer. Functional element 59 can comprise a sensor and/or a transducer. In some embodiments, functional element 59 comprises a sensor configured to produce a signal related to the proper operation of console 50. In some embodiments, functional element 59 comprises an element selected from the group consisting of: an accelerometer; a gyroscope; a pressure sensor; a flow meter; a source of pressurized fluid, such as insufflation gas; a pumping assembly for delivering and/or removing insufflation and/or other fluids; and combinations of one or more of these. In some embodiments, console 50 includes one or more conduits 51 (e.g. wires, optical fibers, fluid tubes, and the like) configured to operably attach console 50 to base unit 600.

Base unit 600 can include a processor 610, including one or more algorithms, algorithm 615, for execution by processor 610, which enables the operation of articulating probe system 10. Processor 610 can further include an image processing unit 616 for receiving and processing optical signals (e.g. signals received from one or more cameras) and/or other image data. In some embodiments, base unit 600 includes an electrical connector port, connector 642, configured to operably connect with a corresponding electrical connector, connector 112 of feeder 100. Electrical connectors 642,112 can comprise physical, optical, near field, RFID, magnetic, and/or other electrical connectors. In some embodiments, base unit 600 and/or console 50 is configured to identify (e.g. via electrical connector 642) one or more properties of an attached feeder 100. For example, base unit 600 and/or console 50 can identify a property selected from the group consisting of: the type of feeder 100 attached, for example a feeder intended for colorectal and or ENT use; a serial number; the number of procedures the feeder has been used in; other operational parameters of the feeder; and combinations of one or more of these. In some embodiments, console 50 and/or base unit 600 modifies and/or reconfigures one or more system parameters based on the identified properties of the attached feeder, such as to enable or disable specific modes of operation. Additionally, base unit 600 can include a camera connector port, camera port 643, configured to operably connect with a corresponding camera connector 383 of a camera assembly 380, as described herein. Base unit 600 can comprise one or more manipulation assemblies 660, manipulation assemblies 660a,b shown, configured to advance, retract, steer, and/or otherwise manipulate articulating probe 300.

Manipulation assembly 660a includes one or more gears 661 configured to engage with corresponding bobbins 111 of feeder 100. Steering cables 311,351 are each operably attached to a bobbin 111 (e.g. wrapped around), such that rotation of a bobbin 111 causes the tightening or loosening (pay out or take up) of the respective cable. Gears 661 are independently driven by manipulation assembly 660a, as instructed by processor 610, in response to one or more user inputs, to cause the rotation of bobbins 111, and subsequent manipulation of probe 300. Manipulation assembly 660b includes connectors 667,669 configured to engage with corresponding projections 317, 359 of feeder 100, respectively. Connectors 667 and 669 are independently linearly translated by manipulation assembly 660b, such as along one or more lead screws or other linear translation assemblies, as instructed by processor 610, in response to one or more user inputs, to cause the advancement of inner probe 310 and/or outer probe 350. In some embodiments, retraction of connector 667 and/or 669 causes the respective retraction of probe 310 and/or 350. Alternatively, connectors 667,669 provide a contact “pushing” force, but do not lockingly engage with projections 317,359, respectively, and a combination of retracting connectors 667,669 and tightening of the cables 311,351 is required to retract inner and outer probes 310, 350, respectively.

Base unit 600 can further include one or more alignment pins 644 constructed and arranged to align corresponding recesses 114 of feeder 100. Alignment pins 644 and recesses 114 can be employed at one or more locations along the interface between base unit 600 and feeder 100, such as to ensure alignment and stability between the two. In some embodiments, an alignment pin 644a is positioned adjacent electrical connector 642, and recess 114a is positioned adjacent electrical connector 112 of feeder 100, such as to ensure the alignment of connectors 642 and 112 when feeder 100 is operably attached to base unit 600. In some embodiments, recess 114a is positioned such that it aligns with a corresponding recess in electrical connector 112, such that alignment pin 644a engages electrical connector board 112 directly.

Introducer 200 can comprise a distal portion of feeder 100, and/or it can comprise a separate component that can be removably attached to a distal portion of feeder 100. Introducer 200 surrounds (i.e. defines) a chamber 205. Chamber 205 can comprise a pressurizable chamber, as described herein. Articulating probe 300 can extend through feeder 100 and into chamber 205, and exit the distal end of introducer 200. Feeder 100 can comprise one or more sealing elements, providing a seal between chamber 205 and the proximal portion of feeder 100, and establishing the proximal seal location for the surgical system when in use. Feeder 100 can comprise a sealed opening 213, which slidingly receives probe 300, and maintains a seal around probe 300 by sealing around sheath 352. In some embodiments, sealed opening 213 maintains a seal directly around probe 300 (e.g. without sheath 352). Introducer 200 can comprise one or more additional sealed openings, each configured to slidingly receive one or more elongate elements, such as sealed opening 223 shown. Sealed opening 223 can be configured to slidingly receive a cable, cable 382 of camera assembly 380, such as to allow camera cable 382 to translate through the proximal seal location while maintaining a seal. Alternatively or additionally, sealed opening 223 can be configured to slidingly receive an elongate element selected from the group consisting of: an electrical conduit; a fluid conduit; an optical fiber; a surgical tool; and combinations of one or more of these. In some embodiments, sealed openings 213, 223, and or other sealed openings described herein configured to slidingly receive an elongate object can comprise an element configured to at least one of increase the seal effectiveness and/or promote the translation of the elongate element through the opening (e.g. reduce friction). In some embodiments, this element can comprise a projection into (e.g. around the perimeter of) the sealed opening, such as an O-ring, a flexible flange, or the like. In some embodiments, this element can comprise a lubricious material, such as a silicone material with an impregnated lubricant.

Introducer 200 can include one or more guide tube access ports, ports 263, with attached connector 264 (263a,b and 264a,b shown), each port 263 configured to receive a tool guide, tool guides 510a,b, each comprising a rigid tube and a fluid tight proximal end. Connectors 264a,b can each comprise a flexible connector, such as to allow flexible positioning of tool guides 510a,b relative to introducer 200. Connectors 264a,b provide a seal to each tool guide 510a,b such as to maintain a seal about chamber 205. Tool guides 510a,b are slidingly received by connectors 264a,b, and projections from connectors 264a,b, projections 266a,b, maintain a seal between connectors 264a,b and tool guides 510a,b, respectively. Tool guides 510a,b can each comprise a proximal sealing element, seal assembly 530a,b, such as a duck bill valve or other sealing element configured to slidingly receive an elongate object while maintaining a seal around the object. Tool guides 510a,b can each slidingly receive a flexible tube, guide tubes 377a,b, operably attached to the distal end of probe 300, for example when guide tubes 377a,b are attached to a portion of a distal link assembly 370, working port 376a,b. Guide tubes 377a,b are attached to working ports 376a,b such that an elongate object, such as a flexible surgical tool, tool 20, can be slidingly received by a guide tube 377 and exit working port 376. Tool 20 can comprise a diameter of less than 6 mm, less than 4 mm, or less than 3 mm.

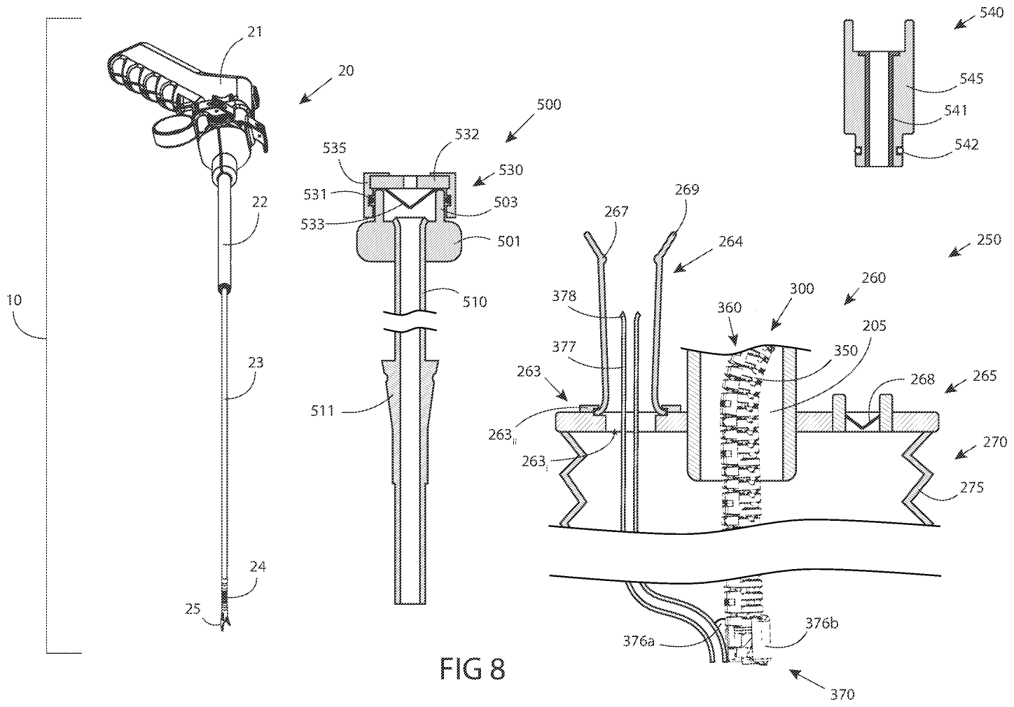

At least a portion of each of guide tubes 377a,b can translate within tool guides 510a,b, such that when probe 300 is advanced (e.g. away from tool guides 510a,b), the proximal ends of guide tubes 377a,b translate distally within tool guides 510a,b, respectively, and when probe 300 is retracted (e.g. towards tool guides 510a,b), the proximal ends of guide tubes 377a,b translate proximally within tool guides 510a,b, respectively. Tool guides 510a,b are each configured to also slidingly receive a tool 20. A tool 20 that is inserted into a tool guide 510, and through sealing assembly 530, is slidingly received by guide tube 377 (which is also slidingly received by tool guide 510). Tool 20 exits guide tube 377 at working port 376, which can be positioned at the distal end of probe 300. Tool 20, tool guides 510a,b, sealing assembly 530, and/or guide tube 377 can be constructed and arranged as is described herebelow in reference to FIG. 8. Tool 20 can comprise a handle 21, operably attached to a support segment 22, generally a rigid tube, configured to be slidingly received by a tool guide 510 such as to provide a rigid pivot point for the manipulation of handle 21. Tool 20 can further comprise a flexible shaft portion, flexible shaft 23, configured to be slidingly received by guide tube 377, conforming to the shape of the channel, and terminating in an articulating portion 24, which is operably attached to an end effector 25. At least articulating portion 24 and end effector 25 can be configured to exit working port 376 and perform a surgical task (e.g. grasp, cut, ablate, coagulate and/or otherwise effect tissue), within an insufflated area. System 10 can comprise one or more tools 20. The one or more tools 20 can comprise flexible tools and/or rigid tools. Tools 20 can comprise articulating portions configured to be manipulated and/or can comprise straight tools configured to operate in as “line of site” from a surgical access point. Tools 20 can comprise various end effectors 25, selected from the group consisting of: a laser delivery element; a scissor; a blade; a cautery element; a suction element (e.g. a nozzle operably connected to a vacuum source); an irrigation element (e.g. a nozzle operably connected to an irrigation source); a grasper; a surgical stapler or other tissue securing element; and combinations of one or more of these.

A distal portion of introducer 200 is configured to operably attach (e.g. in a sealed manner) to body introducer 400. Body introducer 400 can comprise an interior hollow portion, chamber 415, configured to fluidly attach to chamber 205 of introducer 200. Body introducer can further comprise a distal end 410, configured to operably attach (e.g. in a sealed manner) to a body orifice (e.g. a natural and/or surgically created orifice), such that chambers 205, 415 and the body cavity accessed via the body orifice establish a single, sealed environment. Chamber 415 can comprise a pressurizable chamber, as described herein. The single, sealed environment established by chambers 205, 415, and the body cavity can comprise a single, pressurizable chamber, as described herein. Body introducer 400 can comprise components configured to operably connect to an insufflation source and provide insufflation within chamber 415, insufflation port assembly 420. Insufflation port assembly 420 can comprise a luer or other connector configured to attach to a source of insufflation fluid, and a lumen from the connector (outside of chamber 415) to a location inside of chamber 415, such as a location proximate the distal end 410 of body introducer 400. In some embodiments, body introducer 400 further comprises a tool port, 465, operably attached to a lumen 466, each configured to slidingly receive an elongate element, such as a surgical tool with at least a flexible portion. Tool port 465 can comprise a valve, configured to seal tool port 465, and/or to seal around an elongate object positioned within tool port 465. In some embodiments, lumen 466 comprises a lumen within a wall of body introducer 400. The distal end of lumen 466 can be positioned proximate the distal end of body introducer 400, such that an inserted flexible tool exits lumen 466 proximate the distal end of body introducer 400. In some embodiments, lumen 466 comprises a curved or otherwise non-linear geometry.

Camera assembly 380 of system 10 comprises, at its distal end, a camera 381, operably attached to an image data conduit (e.g. a fiber optic conduit and/or a power and/or data carrying conduit), cable 382, terminating in connector 383, and configured to operably attach to camera port 643 of base unit 600. Camera 381 can be operably attached to distal link assembly 370, and cable 382 can be positioned along at least a portion of the length of probe 300. In some embodiments, cable 382 is removably attached to probe 300 with one or more attachment mechanisms, clips 361.

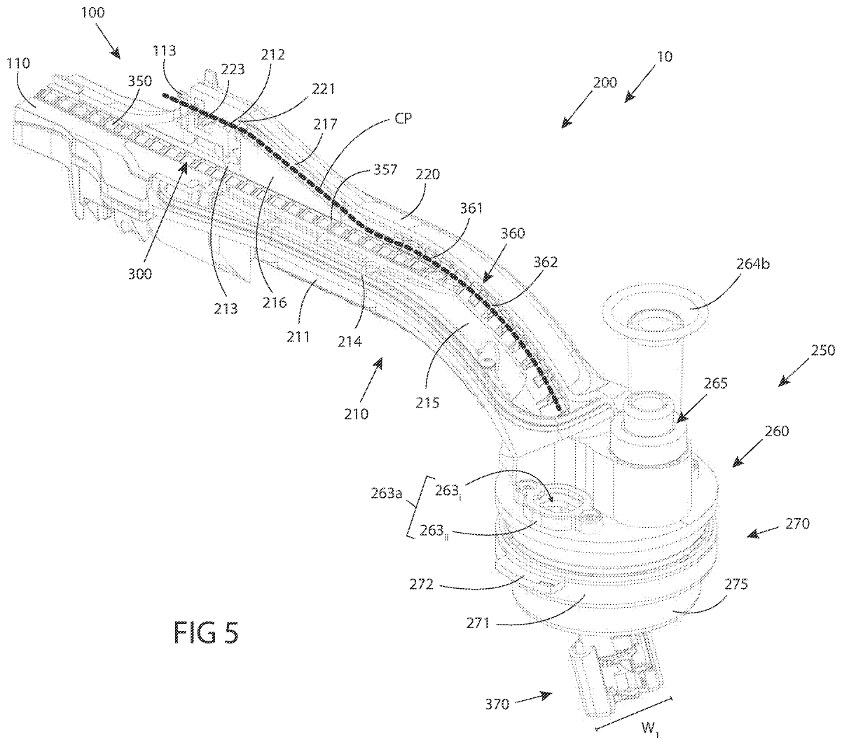

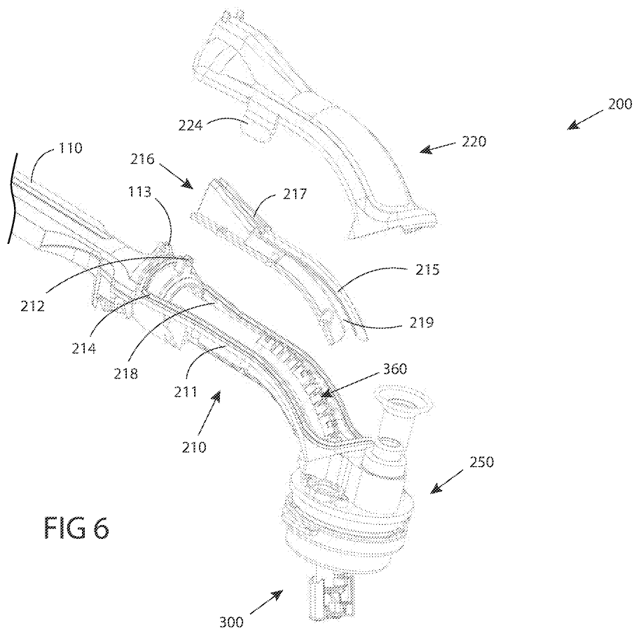

In some embodiments, introducer 200 comprises a housing 210, with a relatively large opening, and a cover 220 configured to attach to housing 210 and seal the opening, providing a seal about chamber 205. Introducer 200 can be constructed and arranged as described in detail herebelow in reference to FIGS. 5 and 6. Cover 220 can be removed from housing 210 to allow access to chamber 205 and/or to allow components to be installed within or inserted through introducer 200, after which cover 220 can be reattached to form chamber 205. Introducer 200 can comprise one or more (two shown) tool ports, tool ports 265, each configured to slidingly receive an elongate tool, shaft or other elongate object, while maintaining a seal about the object.

In some embodiments, system 10 comprises one or more sensors, transducers, and/or other functional elements such as functional elements 199, 299, 399, and/or 499 shown. Functional element 199 comprises one or more functional elements positioned in one or more chambers of feeder 100. Functional element 299 comprises one or more functional elements positioned in one or more chambers of introducer 200. Functional element 399 comprises one or more functional elements positioned in one or more chambers of probe 300. Functional element 499 comprises one or more functional elements positioned in one or more chambers of body introducer 400. In some embodiments, functional elements 199, 299, 399, and/or 499 comprise a sensor configured to produce a signal related to the status of insufflation and/or the status of tissue treatment. In these embodiments, functional elements 199, 299, 399, and/or 499 can comprise one or more sensors selected from the group consisting of: a pressure sensor such as a pressure sensor configured to measure insufflation pressure or a surrogate of insufflation pressure; a light sensor such as a light sensor configured to detect smoke; a position transducer such as an accelerometer or gyroscope configured to track the position of the distal or other portion of probe 300; an accelerometer; a flow sensor; and combinations of one or more of these.

In some embodiments, a sterile barrier can be provided between one or more portions and/or components of system 10, such as is described in reference to applicant's co-pending U.S. Provisional Application Ser. No. 62/504,175, filed May 10, 2017, the contents of which are incorporated herein by reference in their entirety for all purposes.

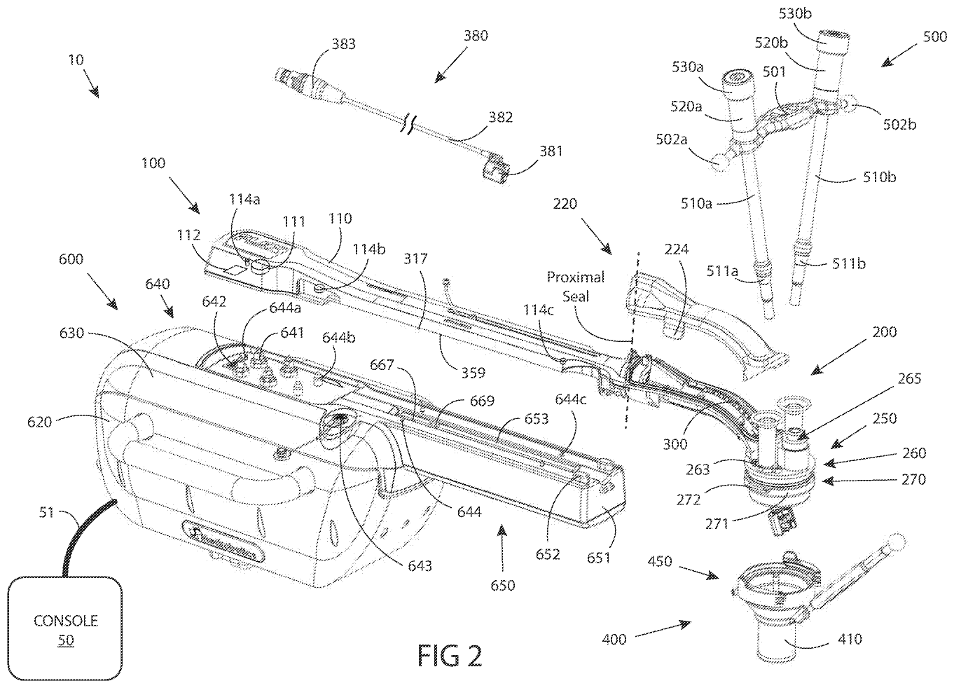

Referring now to FIG. 2, an exploded perspective view of an articulating probe system is illustrated, consistent with the present inventive concepts. Articulating probe system 10 includes a console 50, a base unit 600, and a feeder 100. Feeder 100 can comprise an articulating probe 300. Articulating probe system 10 can further include an introducer 200, a body introducer 400, a camera assembly 380, and an instrument support 500. The components of system 10 can be constructed and arranged as described in reference to PCT Application No. PCT/US16/28374 filed Apr. 20, 2016, the contents of which are incorporated herein by reference in their entirety for all purposes, and can be of similar construction and arrangement to the similar components as described hereabove in reference to FIG. 1. Each of the components of system 10 of FIG. 2 can comprise one or more sealing elements, such as to support an insufflation procedure.

In some embodiments, base unit 600 comprises a base assembly 620 and a top assembly 630 that is removably attached to base assembly 620. Additionally, a first top assembly 630 can be replaced with another, such as a second top assembly 630′, after one or more uses, such as in a disposable (e.g. single use) or resposable (e.g. multiple but limited use) manner. A “use” may include a single procedure performed on a single patient and/or multiple procedures performed on the same and/or multiple patients. In some embodiments, base assembly 620 and top assembly 630 are fixedly attached to one another (e.g. in a manufacturing process). Top assembly 630 can include a proximal portion 640, and a portion extending distally (e.g. towards the patient) from proximal portion 640, extension 650 with a distal end 651. Feeder 100 can be configured to removably attach to base unit 600, and can be replaced with another or second feeder 100′, after one or more uses. In some embodiments, camera assembly 380 is configured to be used in two or more procedures (e.g. assembly 380 is cleaned and/or sterilized between procedures) and is removably installed to probe 300 and attached to base unit 600 at camera port 643 prior to use.

Base unit 600 includes a manipulation assembly 660a, such as is described hereabove in reference to FIG. 1, comprising one or more motors, gears, cables, circuitry, guide rails, motion transfer components, and/or other mechanical or electrical devices that communicate with feeder 100 to control the movement of articulating probe 300, and/or one or more tools 20 of system 10 (e.g. tools 20 positioned within, alongside or otherwise used with articulating probe 300). For example, base unit 600 can include a motor, cable control assembly, or the like (not shown) that drives a carriage assembly within extension 650, which in turn controls a movement (e.g. advancement and/or retraction) of articulating probe 300. The carriage assembly(s) can comprise connectors 667, 669, configured to translate linearly within extension 650. Extension 650 can comprise an opening along the top of its length, slot 653, configured to receive projections 317,359 of probe 300, such that connectors 667,669 can engage projections 317,359, respectively, within extension 650.

As shown in FIG. 2, proximal portion 640 can comprise one or more drive mechanisms, capstans 641, configured to mate with one or more bobbins 111 of feeder 100. Capstans 641 drive steering cables that are actuatable by a cable control assembly, which controls the steering and locking of the inner and outer links of inner and outer probe 310, 350, respectively. The steering cables can be used to releasably tighten to lock (e.g. transition to rigid mode) either or both of the plurality of inner links or the plurality of outer links of articulating probe 300.

In some embodiments, proximal portion 640 includes one or more alignment pins 644b constructed and arranged to align with corresponding recesses 114b of housing 110 of feeder 100. Alignment pins 644 and the corresponding receiving recesses 114 of housing 110 can be employed at one or more locations along the length of top assembly 630 and extension 650 to ensure alignment and stability with feeder 100 when attached to base unit 600. For example, alignment pins 644b can be employed at one or more locations on proximal portion 640 and alignment pins 644c can be employed at one or more locations on extension 650, as shown. Recesses 114b,114c can be positioned along the length of feeder 100 to correspond to alignment pins 644b, 644c. In some embodiments, extension 650 includes one or more release mechanisms, quick release buttons 652, near distal end 651, configured to release feeder 100 from base unit 600. The depression of quick release buttons 652, also referred to as emergency release buttons, disconnects feeder 100 from base unit 600.

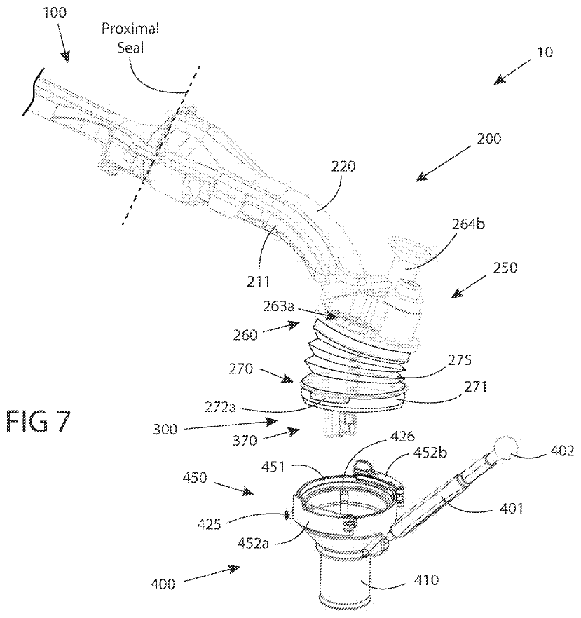

Introducer 200 can be configured to removably attach to the distal end of feeder 100, and articulating probe 300 can extend from feeder 100 and through introducer 200. Introducer 200 includes a cover 220 and a connector 250. Cover 220 can comprise one or more retention elements, clip 224 shown, to releasably secure cover 220 to at least a portion of introducer 200, such as to cover an opening in introducer 200 and form/maintain chamber 205 within introducer 200 as described herein. Connector 250 comprises an upper portion 260 and a lower portion 270. Upper portion 260 can be fixedly attached to the housing of introducer 200, and provide a platform for locating one or more access ports, such as one or more ports 263 and/or tool ports 265. Lower portion 270 can comprise one or more rings 271, one or more projections 272, and one or more bellows 275 (such as those shown in an expanded state in FIG. 7). Bellows 275 can comprise a first end sealed to upper portion 260 and a second end sealed to lower portion 270. Lower portion 270 can be constructed and arranged to articulate relative to upper portion 260, via bellows 275, and to removably attach to body introducer 400, such as is described in further detail herebelow in reference to FIG. 7. Body introducer 400 includes a connector portion, connector 450, with a distal end 410.

Instrument support 500 can comprise one or more tool guides 510 (tool guides 510a,b shown). In some embodiments, a support brace 501, also referred to as a “dogbone connector”, is coupled between the proximal ends of one or more tool guides, 510a,b shown. Support brace 501 can be constructed and arranged to maintain a relative position between tool guides 510a,b. As shown in FIG. 2, tool guides 510a,b can each comprise a guide extender 520, guide extenders 520a,b shown, configured to extend the length of the tool guide, such as is described in detail herebelow in reference to FIG. 8. Tool guides 510a,b can connect to ports 263 of introducer 200 such as is described hereabove in reference to FIG. 1, and herebelow in reference to FIG. 8.

In some embodiments, instrument support 500 includes one or more attachment points 502 (attachment points 502a,b shown) constructed and arranged to removably attach instrument support 500 to a stabilizing brace (not shown). The stabilizing brace can be further attached to other locations related to articulating probe system 10, such as an operating room floor, the patient operating table, and/or feeder 100.

Referring additionally to FIG. 3, perspective views of an articulating probe and a camera assembly for an articulating probe system are illustrated, consistent with the present inventive concepts. Articulating probe 300 is shown removed from feeder 100 for illustrative clarity. Each of the components of probe 300 of FIG. 3 can comprise one or more sealing elements, such as to support an insufflation procedure.

Probe 300 comprises inner probe 310, slidingly received within outer probe 350. Inner probe 310 can comprise a proximal segment 316 (e.g. an elongated proximal link), operably attached to the proximal-most articulating link 315 (link not shown) of inner probe 310. Inner proximal segment 316 can comprise a length such that segment 316 remains within a straight portion of housing 110 as probe 300 is extended from housing 110. Outer probe 350 can also comprise a proximal segment (e.g. an elongated proximal link), 356, operably attached to the proximal-most articulating link 355p, and comprising a length such that segment 356 remains within a straight portion of housing 110 as probe 300 is extended from housing 110. Inner proximal segment 316 and outer proximal segment 356 each comprise lengths such that inner segment 316 does not extend beyond the distal end of outer proximal segment 356 during the operation of probe 300. Projections 317, 359 each comprise projections from inner and outer proximal segments 316,356, respectively.

Proximal segment 356 can comprise an attachment point such as a depression, recess 357, where sheath 352 is sealed and fixedly attached near its proximal end to outer probe 350. In some embodiments, sheath 352 can be adhesively attached (e.g. glued) to recess 357, and/or sheath 352 can comprise a resilient (e.g. elastic) material configured to “self-seal” within recess 357. In some embodiments, the medial portion of sheath 352 slidingly receives outer probe 350, such that sheath 352 and links 355 of outer probe 350 are free to move relative to each other, and sheath 352 minimally affects the articulation of links 355 of outer probe 350. Sheath 352 can comprise a durometer of less than 90 A, such as less than 85 A, or less than 80 A, such that it minimally affects the articulation (e.g. steering) of links 355 of outer probe 350. Sheath 352 can comprise a thickness of less than 0.005″, such as less than 0.004″ or approximately 0.003″. In FIG. 3, probe 300 is in the same position and orientation as shown in FIG. 2 (e.g. a position fully retracted into feeder 100), and the relative position of the proximal seal location is indicated. Introducer 200 seals around sheath 352 of probe 300 to allow for the translation of probe 300 within feeder 100 while maintaining a seal about chamber 205 of introducer 200. The position of recess 357, relative to the proximal seal location, defines an allowable travel distance for probe 300 from its fully retracted position, to an advanced position, in which the seal around sheath 352 is maintained. The proximal end of sheath 352 can be positioned within feeder 100 when articulating probe 300 is advanced to a maximum travel distance. In some embodiments, processor 610 prevents probe 300 from extending beyond the allowed travel distance. Alternatively or additionally, recess 357 can be positioned at a sufficiently proximal location on outer proximal segment 356 that the physical advancement limit of system 10 is shorter than the travel distance allowed by the seal.

Outer probe 350 can comprise, along at least a distal portion, a cable management mechanism, for example “clip strip” 360, comprising a plurality of clips 361a,b, each comprising a recess 362, configured to axially receive and removably attach to cable 382 of camera assembly 380. In some embodiments, the plurality of clips comprises one or more retention clips, clips 361a, comprising a stiff material, configured so secure cable 382 to probe 300. The plurality of clips can also comprise one or more alignment clips, clips 361b, comprising a more flexible material (i.e. more flexible than clips 361a), configured to align cable 382 to probe 300 between retention clips 361a (clips 361a and 361b referred to collectively herein as clips 361). Prior to, during, and/or between surgical procedures, camera assembly 380 can be installed onto probe 300. Camera 381 can be removably attached to distal link assembly 370 of outer probe 350 (as shown), and cable 382 can be attached along at least a portion of the length of probe 300 to clip strip 360 (also as shown). Clip strip 360 is configured to maintain the position of cable 382 relative to probe 300 and to help prevent or at least limit unwanted “slack” in the cable from bunching or otherwise taking an unwanted orientation near the distal portion of probe 300 and preventing or at least limiting damage to cable 382. In some embodiments, clip strip 360 is fixedly attached to sheath 352. In some embodiments, outer proximal segment 356 of outer probe 350 comprises one or more retention mechanisms, clip 358 shown, for maintaining the position of cable 382 relative to a portion of probe 300.

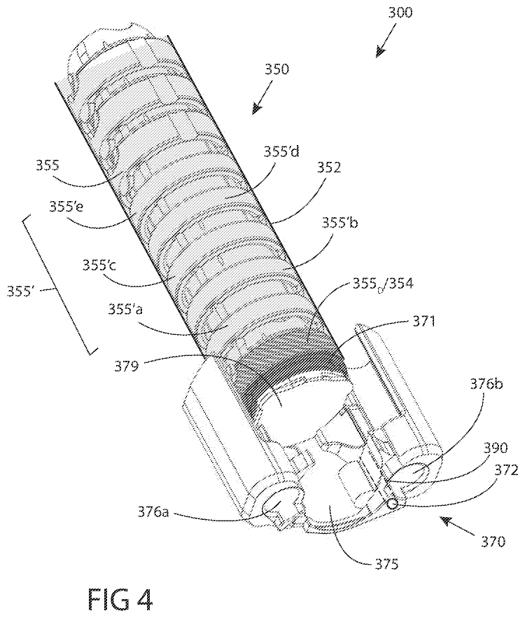

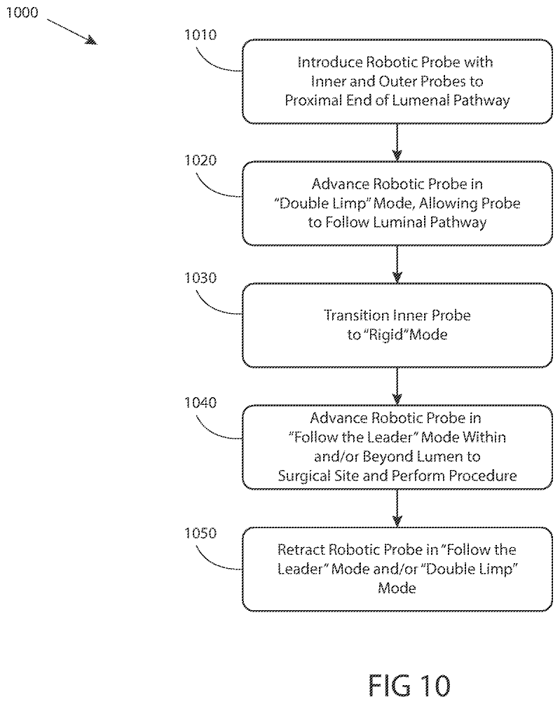

Referring additionally to FIG. 4, a perspective view of the distal end of an articulating probe for an articulating probe system is illustrated, consistent with the present inventive concepts. Clip strip 360 and camera assembly 380 have been removed for illustrative clarity. Each of the components of probe 300 of FIG. 4 can comprise one or more sealing elements, such as to support an insufflation procedure. The distal end of outer probe 350 comprises one or more modified distal links 355′. In the embodiment shown in FIG. 4, modified distal links 355′a-e comprise five links that each have a length (i.e. a length along the axis of probe 300) that is less than the length of a more proximal link 355 (e.g. modified distal links 355′a-e are shorter than intermediate links 355). The smaller length of one or more distal links 355′a-e allows the distal portion of probe 300 to achieve a smaller turning radius (i.e. a smaller achievable radius of curvature of the distal portion of probe 300 corresponding to a maximally articulated orientation), such as to accommodate tortuous pathways to one or more target locations (e.g. the small and large intestine) in the patient. Links 355 and distal links 355′ can be constructed and arranged as described herebelow in reference to FIG. 12.

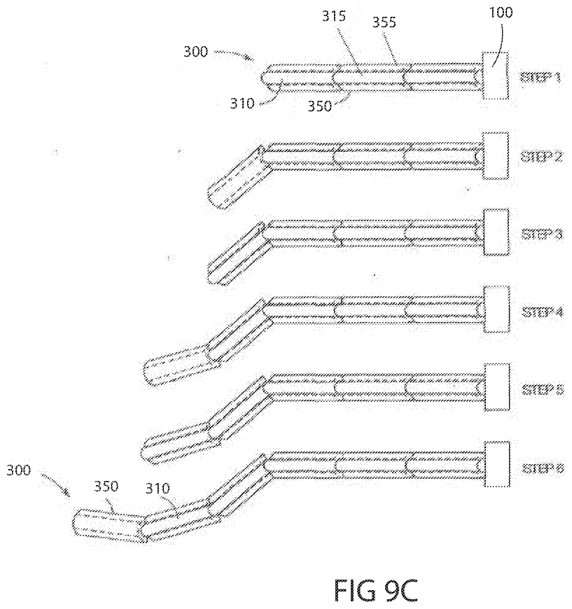

Distal link assembly 370 can comprise a camera housing, housing 375, in which camera 381 can be positioned and secured. Camera 381 is shown secured in housing 375, with its lens oriented in a distal direction. Sheath 352 can be sealed to a distal-most link 355D of probe 300 and/or to housing 375, to provide the sealed configuration of probe 300. Seal 354 can comprise sheath 352 adhesively attached to distal-most link 355D, and/or a compression seal between sheath 352 and link 355o. In the embodiment shown, housing 375 comprises a solid proximal wall (e.g. a sealing surface), proximal wall 379, distal link 355o is sealed to housing 375 by sealing element 371 (e.g. a glue or filler type sealing element), and sheath 352 is sealed to distal link 355D, such that sheath 352, distal link 355D, sealing element 371, and proximal wall 379 function as distal sealing elements and establish a seal around the distal end of probe 300. Sheath 352, along with these distal sealing elements, define a chamber within which probe 300 resides, and is segregated from its insufflated surroundings. In some embodiments, probe 300 comprises one or more internal working channels, such as working channels 390 (not shown but such as are described herein in reference to FIGS. 3 and 11), positioned between outer probe 350 and inner probe 310. Working channels 390 can exist within the chamber defined by sheath 352, such that working channels 390 are segregated from the insufflated surroundings. In some embodiments, a tool or other elongate structure can be slidingly received by a working channel 390, and translated along the length of probe 300, to distal link assembly 370. Conduit 30 can be operably attached to a portion of distal link assembly 370, for example opening 372. Conduit 30 can be configured to deliver one or more tools or other elongate elements to the distal end of probe 300, such as a tool 20. Alternatively or additionally, conduit 30 can supply and/or remove one or more fluids to and/or from the distal end of probe 300 (and as such to the surgical site), such as to provide insufflation and/or desufflation, and/or to deliver irrigation fluid. In some embodiments, sealing of conduit 30 from the insufflated environment is provided via a valve (e.g. located within conduit 30 or at either end). Alternatively or additionally, sealing of conduit 30 can be achieved by placing an elongate element (e.g. a tool) in conduit 30, the elongate element taking up sufficient space of conduit 30 to sufficiently occlude conduit 30 (e.g. by providing sufficient resistance to air or other fluid such as to prevent or at least limit significant passage of the fluid through conduit 30). When conduit 30 (e.g. its proximal end), or other conduit of system 10, is attached to a source of insufflation fluid, a complete seal is not desired, as insufflation fluid is intended to be delivered to the insufflated environment (e.g. to exit conduit 30 to the insufflated environment). During delivery of insufflation fluids, a valve (as described above) can open (e.g. a valve within or proximate conduit 30) through which the insufflation fluid is delivered.

Distal link assembly 370 can include one or more working ports, ports 376a,b shown, each configured to attach to a working channel, removed for illustrative clarity, but as described herein.

Referring additionally to FIG. 5, a cutaway perspective view of an introducer for an articulating probe system is illustrated, consistent with the present inventive concepts. Introducer 200 comprises a housing 210, a cover 220, and a connector 250. Each of the components of system 10 of FIG. 5 can comprise one or more sealing elements, such as to support an insufflation procedure. Housing 210 can comprise multiple discrete components. Introducer 200 can include a sealed opening 213 configured to surround and slidingly receive probe 300, including sheath 352 (sheath 352 not shown for illustrative clarity but surrounding probe 300 as described herein) to maintain a seal between introducer 200 and probe 300. Alternatively, probe 300 comprises an internally sealed probe, and sealed opening 213 is configured to surround and slidingly receive probe 300 without sheath 352. Sealed opening 213, and/or sealed opening 223 can each comprise an O-ring, flange, radial inward projection, and/or other feature constructed and arranged to seal to sheath 352 of probe 300. Feeder 100 can comprise one or more cable management elements configured to maintain (e.g. slidingly maintain) the position of camera cable 382. Camera cable 382 has been removed for illustrative clarity, but is positioned and maintained along cable path CP by these cable management elements. In some embodiments, the distal portion of feeder 100 can include a first cable retention clip, clip 113, constructed and arranged to maintain the position of camera cable 382 proximal to sealed opening 223. Distal to clip 113, camera cable 382 passes through sealed opening 223 into chamber 205. Housing 210 can include a second cable retention clip, clip 212, with projections 221, constructed and arranged to maintain the position of cable 382 distal to sealed opening 223. Housing 210 of introducer 200 can include a ramp 216 with a channel 217. Cable 382 can be slidingly positioned within channel 217 such that cable 382 travels (in a proximal to distal direction as shown by path CP) from a location radially offset from probe 300 to a location adjacent to probe 300. The radially offset positioning of sealed openings 213 and 223 allow independent sealing of camera cable 382 and probe 300. Upper portion 260 of connector 250 can comprise one or more ports 263, each of which including an opening 263, and a sealing retention clip, compression ring 263ᵢᵢ, such as is described herebelow in reference to FIG. 8. For example, upper portion 260 can comprise a first port 263a and a second port 263b (port 263b not shown but positioned behind tool port 265). Each port 263a,b can comprise a connector 264a,b, respectively (connector 264a removed for illustrative clarity). Connectors 264a,b can each comprise an elongate tube configured to provide a seal between an inserted element (e.g. guide tube 510 or an elongate tool) and itself. Connectors 264a,b each seal to ports 263a,b, respectively, via compression ring 263ᵢᵢ.

Referring additionally to FIG. 6, an exploded perspective view of an introducer for an articulating probe system is illustrated, consistent with the present inventive concepts. Introducer 200 comprises a multiple-piece housing, housing 210. Each of the components of introducer 200 can comprise one or more sealing elements, such as to support an insufflation procedure. Housing 210 can comprise a bottom portion 211 and a top portion 215, such that top portion 215 is removably attachable to bottom portion 211. In some embodiments, bottom portion 211 and top portion 215 include openings 218, 219, respectively, that allow for access to clip strip 360 and/or probe 300 when cover 220 is removed from introducer 200. In some embodiments, probe 300 is slidingly secured within introducer 200 between top portion 215 and bottom portion 211. Bottom portion 211 can be constructed and arranged to slidingly receive top portion 215, such that top portion 215 is recessed within bottom portion 211 (e.g. top portion 215 fits within bottom portion 211). Bottom portion 211 can include a “racetrack” gasket, gasket 214, that outlines the perimeter of bottom portion 211. Gasket 214 can comprise an elastomeric material. Gasket 214 can be configured to laterally receive cover 220 to provide a seal between cover 220 and housing 210, such as to support an insufflation procedure as described herein. Cover 220 comprises a proximal portion that is constructed and arranged to slidingly receive ramp 216 of top portion 215 when removably attached to housing 210. In some embodiments, cover 220 includes one or more retention elements, clip 224, configured to secure cover 220 to housing 210. In some embodiments, camera cable 382 (not shown), is secured to clip strip 360, laid along ramp 217, and secured within at least one of clips 113 and/or 213, before cover 220 is attached to introducer 220. Sealed opening 223 can be configured to laterally receive cable 382 (e.g. via a “slit” in a sealing element composing sealed opening 223), such that when cover 220 is attached to introducer 200, cable 382 is held in place by at least one clip 113 and/or 213, and cable 382 is laterally received by sealed opening 223. The seal about cable 382 can be formed by compressing at least a portion sealed opening 223 between cover 220 and introducer 200.

Referring additionally to FIG. 7, a perspective view of a flexible connection between an introducer connector portion and a body introducer for an articulating probe system is illustrated, consistent with the present inventive concepts. System 10 comprises feeder 100, introducer 200, probe 300 and body introducer 400 as shown. Each of the components of system 10 can comprise one or more sealing elements, such as to support an insufflation procedure. Bottom portion 270 of connector 250 is shown displaced from upper portion 260, with bellows 275 extended to accommodate the displacement between bottom portion 270 and upper portion 260. Bottom portion 270 can comprise a ring 271 that includes one or more projections 272 configured to mate with body introducer 400 (272a shown, 272b not shown but positioned behind ring 271), such that connector 250 flexibly connects with body introducer 400. Body introducer 400 comprises a proximal connector portion, connector 450, and a distal portion 410. Connector 450 comprises an opening 451 constructed and arranged to receive probe 300. Distal link assembly 370 of probe 300 comprises a width W₁ configured to fit through (e.g. to be advanced through) distal portion 410. Ring 271 of bottom portion 270 can be slidingly received by opening 451 to create a seal between connector 250 and body introducer 400, such as to support an insufflation procedure as described herein. Connector 450 of body introducer 400 includes one or more clips 452, clips 452a,b shown, configured to secure ring 271 to connector 450. Clips 452a,b can transition from a first position (e.g. an unlocked position), configured to receive ring 271, to a second position (e.g. a locked position) configured to engage and secure projections 272.

In some embodiments, connector 450 includes a support arm 401 with an attachment point 402, constructed and arranged to removably attach body introducer 400 to a stabilizing brace (not shown). The stabilizing brace can be further attached to other locations related to articulating probe system 10, such as an operating room floor, the patient operating table, and/or feeder 100.

Referring now to FIGS. 8 and 8A, unassembled and assembled views, respectively of a tool, an instrument support, and an introducer of an articulating probe system are illustrated, consistent with the present inventive concepts. The instrument support and introducer are shown in sectional views. System 10 comprises tool 20, introducer 200, probe 300, and instrument support 500. Tool 20 comprises a handle 21, operably connected to a support segment 22 with a distally extending flexible shaft 23. In some embodiments, the distal portion of flexible shaft 23 comprises an articulating portion 24 with an end effector 25 (e.g. a cutter, grasper, energy delivery element, or other tissue-effecting element). Handle 21 can be configured to manipulate end effector 25 and/or articulating portion 24.

Instrument support 500, introducer 200, probe 300, and other components of system 10 can be of similar construction and arrangement to the similar components described hereabove in reference to FIG. 1 and otherwise as described herein. Each of the components of system 10 can comprise one or more sealing elements, such as to support an insufflation procedure.

Instrument support 500 comprises a tool guide 510 that includes a flange, flange 511, configured to operably attach to connector 264, as described herein. Tool guide 510 can be fixedly attached to one or more other tool guides 510 via a support brace 501, such as is described hereabove in reference to FIG. 2. Tool guide 510 can comprise a flared proximal end, as shown, to ease the insertion of a tool 20. Support brace 501 can include one or more projections 503 (e.g. radial projections), configured to surround the proximal opening of tool guide 510, and provide a sealing surface for slidingly receiving a sealing assembly and/or an extension assembly, as described immediately herebelow. Instrument support 500 can include a seal assembly 530 comprising a housing 535, configured to slidingly receive projection 503, such as to seal the proximal end of projection 503. Seal assembly 530 can comprise a seal O-ring 531 that is configured to surround and/or engage projections 503, such as in a sealing manner. Seal assembly 530 can further comprise a seal 532, and a valve 533. As shown in FIG. 8A, seal 532 and valve 533 are constructed and arranged to slidingly receive support segment 22 and/or shaft 23 of tool 20, such that seal 532 and valve 533 interface with support segment 22 and/or shaft 23 to create a seal.

Instrument support 500 can further comprise an optional extension, adapter 540, configured to be positioned between support brace 501 and seal assembly 530, such as to extend the working length of tool guide 510. Adapter 540 comprises a housing 545, surrounding a lumen, with a distal end configured to be slidingly received within projection 503 of support brace 501. Adapter 540 further comprises a proximal end configured to be slidingly received by seal assembly 530. Adapter 540 can comprise an insert, sleeve 541, configured to provide a lubricious surface to the lumen within adapter 540. Adapter 540 can further comprise an 0-ring 542 configured to engage projections 503 (e.g. and provide a seal).

Upper portion 260 of introducer connector 250 can further include one or more ports 263. As shown, port 263 can comprise an opening 263, and a retention element, compression ring 263n. Connector 264 can be sealed to port 263 via compression of the distal end of connector 264 between upper portion 260 and compression ring 263n. Connector 264 can include a flared proximal end, funnel 269. As shown in FIG. 8A, connector 264 can slidingly receive a tool guide 510 of instrument support 500. Funnel 269 can be configured to extend outward from the proximal end of connector 264, such that connector 264 comprises a funnel shape as shown.

Connector 264 can include a projection 267 configured to frictionally engage a portion of flange 511, such that the interface of projection 267 and flange 511 creates a seal. Guide tube 377 can comprise a flared proximal end, funnel 378, and can be slidingly received by tool guide 510, when tool guide 510 is slidingly received by connector 264. Guide tube 377 extends through opening 263ᵢ, distally through bellows 275, lower portion 270, and body introducer 400 (not shown), and operably connects to working port 376 (port 376a shown), such that a tool 20, inserted into the proximal end of tool guide 510, traverses tool guide 510, into guide tube 377, and exits working port 376.

Upper portion 260 can further include one or more tool ports 265. Port 265 can comprise a valve 268, configured to slidingly receive an elongate element while maintaining a seal about the elongate element. Additionally or alternatively, valve 268 can maintain a seal limiting insufflation fluid from exiting port 265 when an elongate element is not positioned thru port 265.

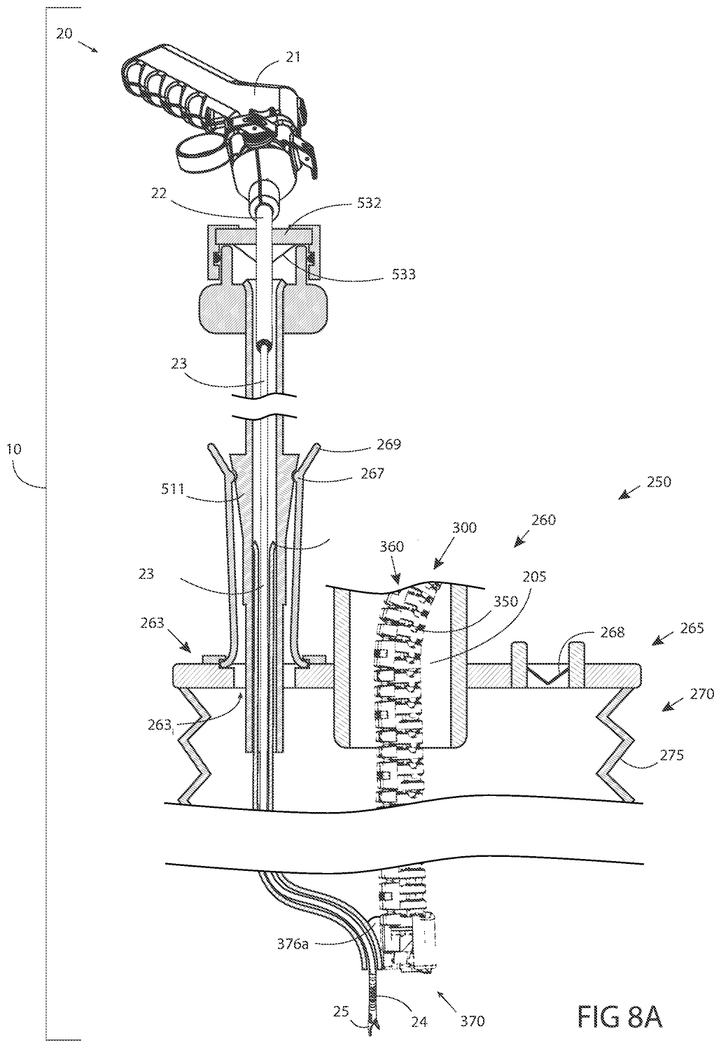

Referring now to FIGS. 9A-C, graphic demonstrations of an articulating probe are illustrated, consistent with the present inventive concepts. Articulating probe 300 comprises essentially two concentric mechanisms, an outer mechanism and an inner mechanism, each of which can be viewed as a steerable mechanism. Each of the components of probe 300 can comprise one or more sealing elements, such as to support an insufflation procedure. FIGS. 9A-C show the concept of how different embodiments of articulating probe 300 operate. Referring to FIG. 9A, the inner mechanism can be referred to as a first mechanism or inner probe 310. The outer mechanism can be referred to as a second mechanism or outer probe 350. Each mechanism can alternate between rigid and limp states. In the rigid mode or state, the mechanism is just that—rigid. In the limp mode or state, the mechanism is highly flexible and thus either assumes the shape of its surroundings or can be re-shaped. It should be noted that the term “limp” as used herein does not necessarily denote a structure that passively assumes a particular configuration dependent upon gravity and the shape of its environment; rather, the “limp” structures described in this application are capable of assuming positions and configurations that are desired by the operator of the device, and therefore are articulated and controlled rather than flaccid and passive.

In some embodiments, one mechanism starts limp and the other starts rigid. For the sake of explanation, assume outer probe 350 is rigid and inner probe 310 is limp, as seen in step 1 in FIG. 9A. Now, inner probe 310 is both pushed forward by feeder 100, and a distal-most inner link 315D is steered, as seen in step 2 in FIG. 9A. Now, inner probe 310 is made rigid and outer probe 350 is made limp. Outer probe 350 is then pushed forward until a distal-most outer link 355D catches up to the distal-most inner link 315D (e.g. outer probe 350 is coextensive with inner probe 310), as seen in step 3 in FIG. 9A. Now, outer probe 350 is made rigid, inner probe 310 limp, and the procedure then repeats. One variation of this approach is to have outer probe 350 be steerable as well. The operation of such a device is illustrated in FIG. 9B. In FIG. 9B it is seen that each mechanism is capable of catching up to the other and then advancing one link beyond. According to one embodiment, outer probe 350 is steerable and inner probe 310 is not. The operation of such a device is shown in FIG. 9C.

In medical applications, operation, procedures, and so on, once articulating probe 300 arrives at a desired location, the operator, such as a surgeon, can slide one or more tools through one or more working channels of outer probe 350, inner probe 310, or one or more working channels formed between outer probe 350 and inner probe 310, such as to perform various diagnostic and/or therapeutic procedures. In some embodiments, the channel is referred to as a working channel that can, for example, extend between first recesses formed in a system of outer links and second recesses formed in a system of inner links. Working channels may be included on the periphery of articulating probe 300, such as working channels comprising one or more radial projections extending from outer probe 350, these projections including one or more holes sized to slidingly receive one or more tools. As described with reference to other embodiments, working channels may be positioned on other locations extending from, on, in, and/or within articulating probe 300.

Inner probe 310 and/or outer probe 350 are steerable and inner probe 310 and outer probe 350 can each be made both rigid and limp, allowing articulating probe 300 to drive anywhere in three-dimensions while being self-supporting. Articulating probe 300 can “remember” each of its previous configurations and for this reason, articulating probe 300 can retract from and/or retrace to anywhere in a three-dimensional volume such as the intracavity spaces in the body of a patient such as a human patient.

Inner probe 310 and outer probe 350 each include a series of links, i.e. inner links 315 and outer links 355 respectively, that articulate relative to each other. In some embodiments, outer links 355 are used to steer and lock articulating probe 300, while inner links 315 are used to lock articulating probe 300. In a “follow the leader” fashion, while inner links 315 are locked, outer links 355 are advanced beyond the distal-most inner link 315D. Outer links 355 are steered into position by the system steering cables, and then locked by locking the steering cables. The cable of inner links 315 is then released and inner links 315 are advanced to follow outer links 355. The procedure progresses in this manner until a desired position and orientation are achieved. The combined inner links 315 and outer links 355 may include working channels for temporary or permanent insertion of tools at the surgery site. In some embodiments, the tools can advance with the links during positioning of articulating probe 300. In some embodiments, the tools can be inserted through the links following positioning of articulating probe 300.

One or more outer links 355 can be advanced beyond the distal-most inner link 315D prior to the initiation of an operator controlled steering maneuver, such that the quantity extending beyond the distal-most inner link 315D will collectively articulate based on steering commands. Multiple link steering can be used to reduce procedure time, such as when the specificity of single link steering is not required. In some embodiments, between 2 and 20 outer links can be selected for simultaneous steering, such as between 2 and 10 outer links or between 2 and 7 outer links. The number of links used to steer corresponds to achievable steering paths, with smaller numbers enabling more specificity of curvature of articulating probe 300. In some embodiments, an operator can select the number of links used for steering (e.g. to select between 1 and 10 links to be advanced prior to each steering maneuver).

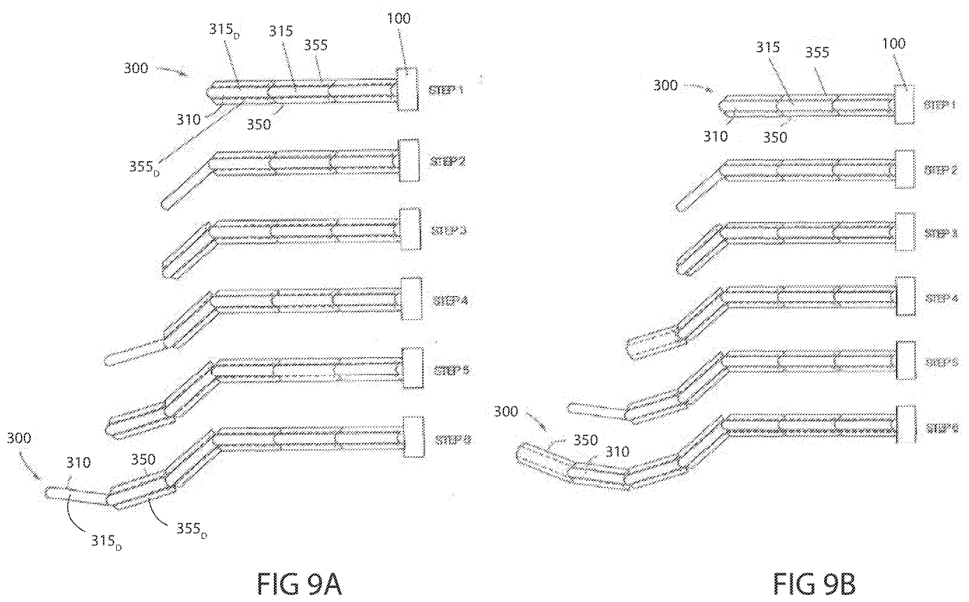

Referring now to FIG. 10, a flow chart of a method for advancing and/or retracting an articulating probe (e.g. probe 300 of system 10 described herein) during a procedure is illustrated, consistent with the present inventive concepts. Method 1000 comprises a series of steps, STEPs 1010-1050 shown, for efficiently advancing and/or retracting an articulating probe at least partially within a luminal pathway. Probe 300 can comprise an inner probe 310 and an outer probe 350, and can be configured to advance in a follow the leader type method as described in reference to FIGS. 9A-C hereabove. Additionally, in the case of advancement through a luminal pathway, or other defined pathway, probe 300 can advance and/or retract in a “double limp” mode, as described immediately herebelow. In STEP 1010, an articulating probe 300 is introduced proximate the proximal end of a luminal pathway, such as the proximal end of the colon. In STEP 1020, articulating probe 300 is advanced within the luminal pathway. Articulating probe 300 is advanced in a double limp mode, such that articulating probe 300 tracks or otherwise follows the luminal pathway (e.g. the pathway of the colon; intestine; esophagus; nasal passageway; vaginal canal; ear canal; and/or other body lumen). In this configuration, both inner and outer probes 310,350 advance simultaneously, both in a limp state, without steering provided by feeder 100. Alternatively or additionally, inner probe 310 can be configured in a rigid mode, and outer probe 350 advanced in a limp mode, with or without steering provided by feeder 100, with the distal portion of outer probe 350 that extends beyond inner probe 310 tracking or otherwise following the luminal pathway. In STEP 1030, inner probe 310 transitions from the “limp” mode to a “rigid” mode (or outer probe 350 is transitioned to a rigid mode, and inner probe 310 is brought forward to “catch up” and then transitions to a rigid mode).

In STEP 1040, articulating probe 300 is advanced in a “follow the leader” mode within the luminal pathway, such as is described hereabove in reference to FIGS. 9A-C. Articulating probe 300 can be advanced within and/or beyond the luminal pathway to a target location, and a surgical procedure can be performed. A surgical procedure can be performed at one or more target locations. In some embodiments, articulating probe 300 is advanced to a first target location and a first procedure is performed, and subsequently, articulating probe 300 is further advanced or retracted to a second target location and a second procedure is performed. In STEP 1050, articulating probe 300 is retracted within the luminal pathway in the follow the leader mode and/or the double limp mode. In some embodiments, articulating probe 300 is retracted in an “enhanced” follow the leader retraction mode such that inner probe 310 is retracted further than in the traditional follow the leader mode (e.g. further than what is used to advance to a target location).

In some embodiments, algorithm 615 of processor 610 comprises one or more algorithms configured to determine the most efficient advance and/or retract “mode” for the commands received from a user. For example, algorithm 615 can be configured to determine the tortuosity of probe 300 (e.g. by “remembering” the steering commands received and calculating the overall shape of the probe when a target location is reached), and compare the tortuosity to a threshold, such as to determine the safety and/or efficiency of retracting in an advanced and/or a double limp mode. In some embodiments, for example when system 10 is being used in a procedure in which probe 300 is near and/or articulated around or behind the heart of the patient, algorithm 615 can prevent double limp advancement and/or retraction (e.g. as described hereabove). Alternatively or additionally, algorithm 615 can be configured to prevent enhanced follow the leader retraction (e.g. also as described hereabove) and/or other enhanced modes (e.g. other enhanced advancement and/or retraction of inner probe 310 and/or outer probe 350 configured to increase speed and/or reduce operator steps).

Referring now to FIG. 11, a cross-sectional view of an articulating probe comprising inner and outer links is illustrated, consistent with the present inventive concepts. Probe 300 comprises multiple outer links 355 and inner links 315, as described herein. Each outer link 355 can comprise one or more recesses 3551, such as the three recesses 3551a-c shown. Inner link 315 can comprise one or more recesses 3151, such as the three recesses 3151a-c shown. Recesses 3551a-c can align with recesses 3151a-c, to form working channels 390a-c shown (generally working channels 390). In some embodiments, probe 300 includes two, four, five, or more working channels 390. Working channels 390 can comprise similar or dissimilar diameters. While shown are relative circles, any of working channels 390 can comprise a non-circular shape, such as an oval or rectangular shape. In some embodiments, any of working channels 390a-c comprise a major axis and/or a minor axis with a length of between 1.0 mm and 12 mm, such as a length between 2.0 mm and 5.0 mm, or a length of approximately 2.5 mm, or approximately 3.3 mm.

Referring now to FIG. 12, cross sectional and side views of proximal and distal outer links of an articulating probe are illustrated, consistent with the present inventive concepts. Outer probe 350 can comprise a plurality of proximal outer links 355, and a plurality of distal outer links 355′. Proximal outer links 355 and distal outer links 355′ can be of similar construction and arrangement to outer links 355,355′ described hereabove. Distal outer links 355′ are constructed and arranged to enable a radius of curvature less than the radius of curvature enabled by proximal outer links 355, such as a radius of curvature of less than or equal to 30 mm, or less than or equal to 20 mm. As shown, proximal and distal outer links 355,355′ each comprise a projecting flange (e.g. a radial projection), shoulders 3552,3552′, respectively, which interfere with the respective flanges of adjacent outer links, such as to limit the enabled minimum radius of curvature of probe 350. Shoulders 3552,3552′ each comprise a shoulder height, heights SH,SH′, respectively. Proximal outer links 355 can comprise a shoulder height SH of approximately 0.21″, and/or an overall height (full height of the outer link) of approximately 0.39″. In some embodiments, distal outer links 355′ can comprise a shoulder height SH′ that is shorter than the shoulder height SH of the proximal outer links 355. For example, the shoulder height SH′ of the distal outer links 355′ can be at least 0.02″ shorter than shoulder height SH of the proximal outer links 355, such as a shoulder height SH′ that is at least 0.03″ shorter, at least 0.04″ shorter, or at least 0.05″ shorter. In some embodiments, distal outer links 355′ comprise a shoulder height SH′ of approximately 0.16″ and/or an overall height of 0.36″ (e.g. while the proximal outer links 355 comprise a shoulder height of approximately 0.21″ and/or an overall height of approximately 0.39″).

Outer links 355,355′ can each comprise a tapered opening, tapers 3554,3554′, respectively. Tapers 3554,3554′ can be constructed and arranged such that the inner walls of links 355,355′ do not interfere with inner probe 310 when probe 300 is in an articulated position (e.g. a minimum radius of curvature corresponding to a maximally articulated orientation), such as is described in reference to applicant's co-pending U.S. patent application Ser. No. 14/587,166, filed Dec. 31, 2014, the contents of which are incorporated herein by reference in their entirety for all purposes. Taper 3554′ of distal outer links 355′ can comprise an angle α′ that is greater than the angle α of taper 3554 of proximal outer links 355, such as a greater angle configured to accommodate a smaller radius of curvature achievable by distal outer links 355′ (as described hereabove). In some embodiments, taper 3554′ of distal outer links 355′ comprises an angle α′ that is at least 2°, at least 5°, or at least 10° greater than the angle α of taper 3554 of proximal outer links 355. In some embodiments, angle α′ is approximately 23.5° and angle α is approximately 13.5°.

Proximal outer links 355 can further comprise one or more anti-rotation features configured to prevent or at least limit axial rotation between adjacent proximal outer links 355. Proximal outer link 355 shown comprises slot 3553, configured to slidingly engage pin 3555, projecting from the articulating surface of an adjacent proximal outer link 355. The engagement of pin 3555 and slot 3553 limits axial rotation of adjacent proximal outer links 355 relative to each other. Links 355 can comprise one or more anti-rotation features similar to features described in reference to applicant's U.S. Pat. No. 9,572,628, filed Mar. 8, 2016, the contents of which are incorporated herein by reference in their entirety for all purposes. In some embodiments, distal links 355′ do not comprise an anti-rotation feature (e.g. due to their limited quantity, and such as to simplify manufacture). In some embodiments, distal outer links 355′ do comprise an anti-rotation feature (not shown), such as a similar or dissimilar anti-rotation feature as proximal outer links 355.

Outer links 355,355′ can comprise one or more recesses, recesses 3551,3551′ shown, respectively, configured to provide (in cooperation with mating recesses of inner links) working channels within probe 300, such as are described hereabove in reference to FIG. 11.

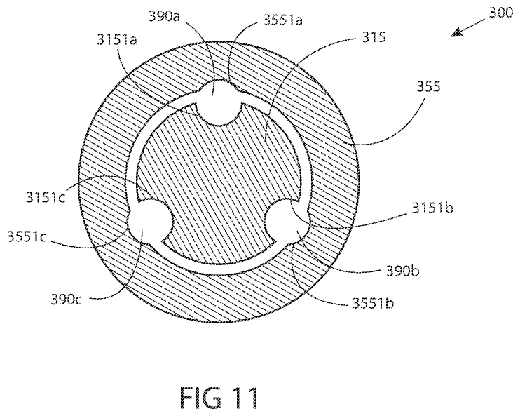

Referring additionally to FIG. 13, a sectional side view of a body introducer is illustrated, consistent with the present inventive concepts. Body introducer 4020 comprises a connector portion 450′, a rigid portion 4128, and a flexible portion 4121. Body introducer 4020 is constructed and arranged to allow access to the body by applying a conformable outward force to achieve desired dilation at the insertion location.

Connector portion 450′ comprises an opening constructed and arranged to receive probe 300. In some embodiments, the connector portion 450′ is constructed and arranged to couple with a bellows 275′. Bellows 275′ can be of similar construction and arrangement to bellows 275 described hereabove in reference to FIG. 7. Bellows 275′ provides a flexible connection between connector portion 450′ and connector 250 of introducer 200, as described herein.

Introducer 4020 can further comprise a flexible portion 4121. Flexible portion 4121 can comprise an access conduit 4122 and an anchoring flange 4123. Distal link assembly 370 of probe 300 comprises a width configured to fit through (e.g. to be advanced through) access conduit 4122.

In some embodiments, flexible portion 4121 is collapsible to allow for easier insertion into the patient. Upon insertion into the body, access conduit 4122 can apply an outward radial force on the body. In some embodiments, access conduit 4122 applies an outward radial force on the sphincter. This application of force allows the muscles to loosen gradually and thereby reduce trauma to tissue of the patient, as opposed to a rigid body introducer which requires dilation of tissue in the process of gaining access.

The distal end of flexible portion 4121 can comprise an anchoring flange 4123. Anchoring flange 4123 maintains the placement of body introducer 4020 within the patient.

The force applied by flexible portion 4121 can be modified by adjusting characteristics of flexible portion 4121, such as by varying materials of construction and/or cross-sectional area. For example, the rigidity of the material can be selected to provide an appropriate resistance to sphincter contraction, while still allowing for collapse during insertion into the patient. The cross-sectional dimensions of flange 4123, access conduit region 4122, and flexible portion 4121 can be selected for different patient parameters, such as body configuration and age and the like. In some embodiments, one or more of flange 4123, access conduit region 4122, and/or flexible portion 4121 comprise a circular cross-section. In some embodiments, one or more of flange 4123, access conduit region 4122, and/or flexible portion 4121 comprise an oval or elliptical cross-section. Similarly, the length L of access conduit region 4122 can be selected to accommodate one or more patient parameters.

In some embodiments, a rigid portion 4128 couples flexible portion 4121 to connector portion 450′. In some embodiments, flexible portion 4121 is sealed to the connector portion 450′, such as to support an insufflation procedure. In some embodiments, rigid portion 4128 is directly connected to flexible portion 4121. In some embodiments, introducer 4020 includes one or more insufflation ports, such as port 4125 shown.

In some embodiments, connector portion 450′ includes a support arm 401 constructed and arranged to removably attach introducer 4020 to a stabilizing brace (not shown). The stabilizing brace can be further attached to other locations related to articulating probe system 10, such as an operating room floor, the patient operating table, and/or feeder 100.

In some embodiments, introducer 4020 comprises a rigid introducer portion (e.g. a standard non-flexible rectoscope) and a flexible portion 4121, each interchangeably attachable to rigid portion 4128 and bellows 275′. In these embodiments, a user of system 10 can choose which introducer portion to use (e.g. rigid or flexible) at the time of a procedure.

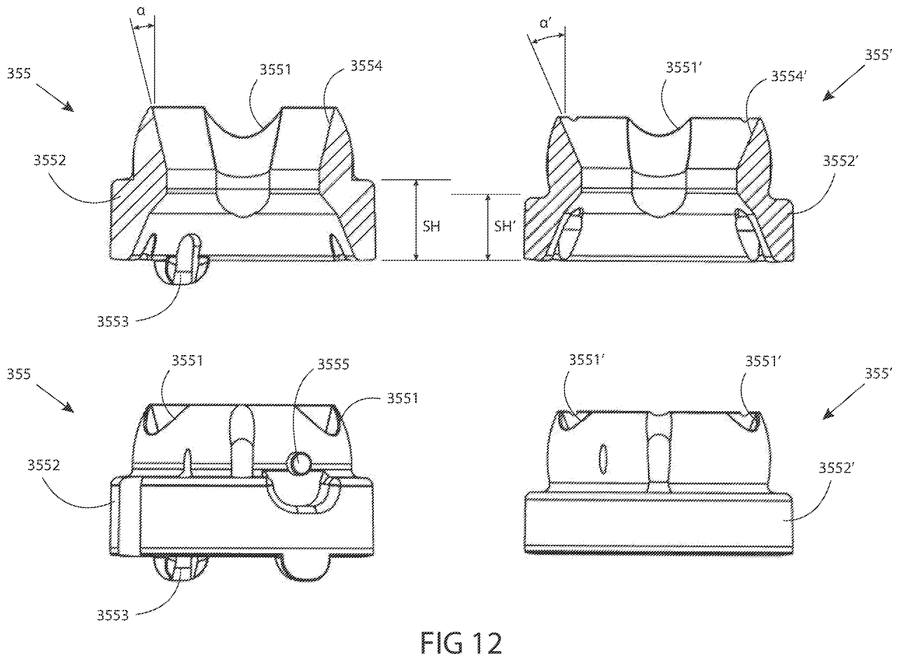

Referring additionally to FIG. 14, a sectional side view of a body introducer and an adapter are illustrated, consistent with the present inventive concepts. Body introducer 400 comprises a connector portion 450 and an access conduit 412. Distal link assembly 370 of probe 300 comprises a width configured to fit through (e.g. to be advanced through) the access conduit 412. Adapter 4130 comprises a conformable sleeve 4132, an anchoring ring 4133, a locking collar 4135, a roller lock 4138, a spring 4137, and a roller 4136. Adapter 4130 is constructed and arranged to allow access to the body by applying a conformable outward force to achieve the desired dilation.

When inserting adapter 4130, the tension on sleeve 4132 between anchoring ring 4133 and roller 4136 is selected to be sufficiently low to allow the orientation of anchoring ring 4133 to be adjusted. In some embodiments, once anchoring ring 4133 is positioned within the patient (for example, positioned on the interior wall of the sphincter), sleeve 4132 can be pulled in the proximal direction (e.g. manually pulled or pulled with mechanical assistance), such that sleeve 4132 passes between roller 4136 and locking collar 4135. As sleeve 4132 is pulled in the proximal direction, sleeve 4132 drives rollers 4136 against spring plate 4137, creating room for sleeve 4132 to pass. During this process, spring plates 4137 can push back on rollers 4136, pinching sleeve 4132 against locking collar 4135, and thereby ensuring tension on sleeve 4132 between anchoring ring 4133 and rollers 4136. In some embodiments, rollers 4136 are arranged to rotate only in a single direction, thereby ensuring that the tension on sleeve 4132 between rollers 4136 and anchoring ring 4133 does not change unexpectedly. In some embodiments, as the tension on sleeve 4132 between rollers 4136 and anchoring ring 4133 increases, the outward radial force on sleeve 4132 increases, causing the diameter of sleeve 4132 to dilate. To release sleeve 4132, locking ring 4135 can be removed, allowing sleeve 4132 to pass over rollers 4136 in the distal direction. In some embodiments, adapter 4130 is configured to seal against a rectoscope or other ports. In some embodiments, the path of rollers 4136 comprises a surface comprising an acute angle to the corresponding surface on locking ring 4135, which pinches sleeve 4132.

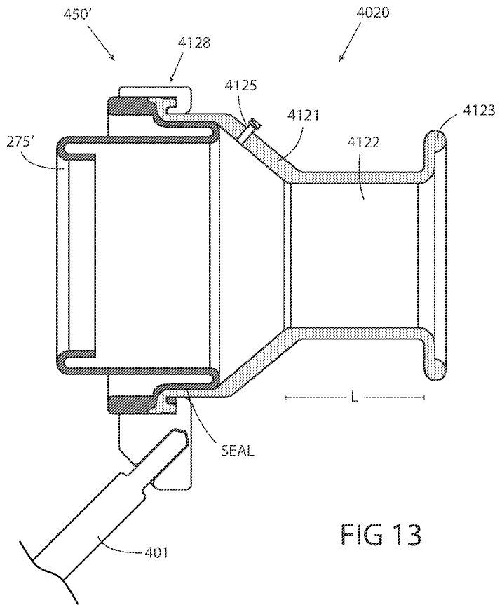

Referring additionally to FIGS. 15A and 15B, a sectional side view and a perspective view of a body introducer and an adapter are illustrated, respectively, consistent with the present inventive concepts. Body introducer 400 comprises a connector portion 450, an access conduit 412, and a projection 417. Distal link assembly 370 of probe 300 comprises a width configured to fit through (e.g. to be advanced through) the access conduit 412.

In the embodiment shown in FIG. 15A and 15B, adapter 4140 comprises a handle 4142, an access conduit 4145, an anchoring flange 4143, a placement feature 4146, and a proximal flange 4141. In some embodiments, adapter 4140 is flexible to allow for easier manual placement into the patient. Anchoring flange 4143, when expanded in the body, maintains the placement of introducer 4140 within the patient. Once introducer 4140 is positioned in the patient, body introducer 400 is inserted further into adapter 4140, toward the distal end of introducer 4140. By inserting body introducer 400 further into adapter 4140, the wall of access conduit 4145 increasingly experiences an outward radial pressure and gradually dilates to the desired diameter. In some embodiments, adapter 4140 comprises one or more placement features 4146. Placement features 4146 can be constructed and arranged to mate with projection 417 of body introducer 400, such as to facilitate placement of body introducer 400.

In some embodiments, body introducer 400 includes a support arm 401, which can be constructed and arranged to removably attach body introducer 400 to a stabilizing brace (not shown). The stabilizing brace can be further attached to other locations related to articulating probe system 10, such as an operating room floor, the patient operating table, and/or feeder 100.

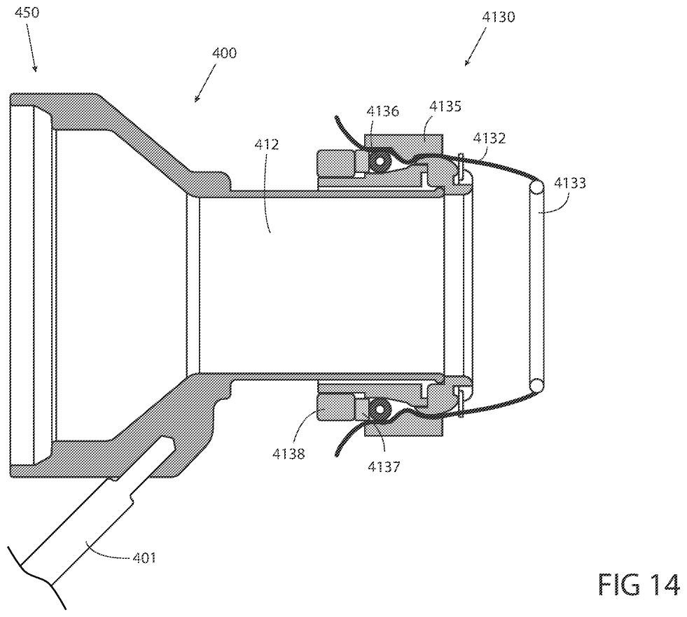

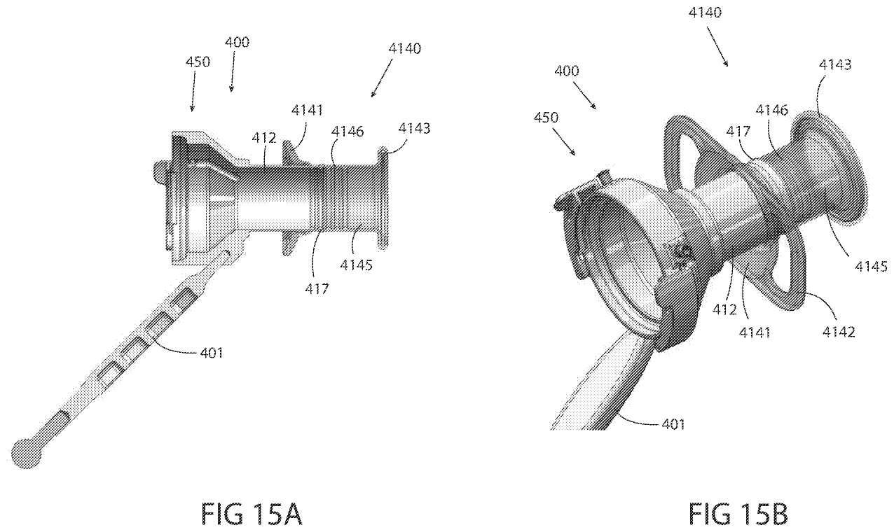

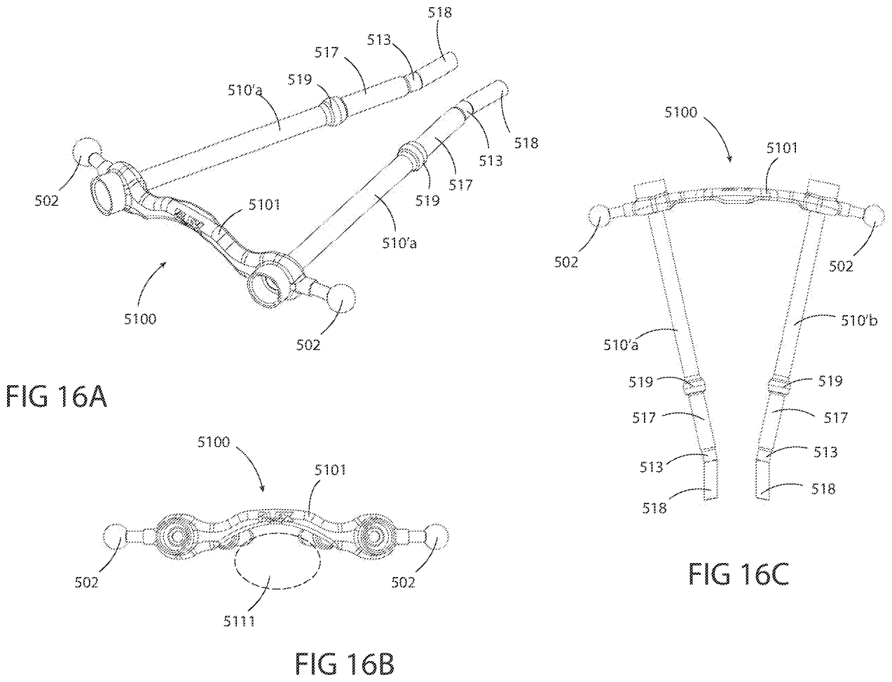

Referring additionally to FIG. 16A, a perspective view of a shaped instrument support is illustrated, consistent with the present inventive concepts. Shaped instrument support 5100 can comprise a shaped brace, brace 5101, and one or more non-linear, or bent, tool guides, tool guide 510′.

Brace 5101, also referred to herein as a “dogbone connector”, can be coupled between the proximal ends of one or more tool guides 510′. Brace 5101 can be constructed and arranged to maintain a relative position between tool guides 510′. As shown in FIGS. 16A-C, brace 5101 can comprise a curved geometry in a plane transverse to an axis of extension of a first tool guide 510′a and an axis of extension of a second tool guide 510′b. The curvature in brace 5101 creates a space 5111 between the tool guides 510′a and 510′b and can allow for easier access to tool port 265 (not shown, but described herein). Space 5111 can allow the user to have greater articulation and maneuverability when using an instrument through tool port 265.

Tool guides 510′ can connect to ports 263 of introducer 200 as previously described. In some embodiments, a distal portion of tool guide 510′ comprises a bend region 513, such that when tool guides 510′ are coupled to instrument support 5101, the distal portions 518 extend along an axis orthogonal to tool ports 265. Tool guides 510′ can each comprise a sealing segment, segment 517, located between a radial projection, proximal stop 519, and bend region 513. Segments 517 can comprise an outer diameter configured to slidingly seal to projection 267 of sealing element 264 (not shown, but described hereabove in reference to FIG. 8). Segment 517 can be configured such that projection 267 can slidingly engage (and seal thereto) segment 517 along its length, such that tool guides 510′ can operably attach to introducer 200 in various positions. The position of tool guides 510′ can be adjusted toward and/or away from tool port 265 (e.g. such that projection 267 engages segment 517 proximate proximal stop 519 or proximate bend 513, respectively). Proximal stop 519 and/or bend 513 can be configured as physical limiter and/or visual markers configured to inform the user of the limits of the positioning. In some embodiments, distal portions 518 extend orthogonally through openings 263i (of FIG. 8) as the position of tool guides 510′ is adjusted.

In some embodiments, instrument support 5100 includes one or more attachment points 502 constructed and arranged to removably attach instrument support 5100 to a stabilizing brace (not shown). The stabilizing brace can be further attached to other locations related to articulating probe system 10, such as an operating room floor, the patient operating table, and/or feeder 100.

Referring additionally to FIG. 16B, a top view of an embodiment of a shaped instrument support 5100 is illustrated, consistent with the present inventive concepts. Space 5111 between tool guides 510′a and 510′b can allow for easier access to tool port 265. Space 5111 can allow the user to have greater articulation and maneuverability when using a rigid laparoscopic instrument through tool port 265.

Referring additionally to FIG. 16C, a side view of an embodiment of a shaped instrument support 5100 is illustrated, consistent with the present inventive concepts.

In some embodiments, the shaped brace 5101 comprises a curved geometry in a plane transverse to an axis of extension of a first tool guide 510′ and an axis of extension of a second tool guide 510′. The curved geometry in the shaped brace 5101 creates a viewing space region 5111 between the first and second tool guides 510′. In some embodiments, the viewing space region 5111 is positioned along a center line taken between the proximal ends of the first and second tool guides 510. At the same time, as shown in the embodiment of FIG. 16B, a portion of the shaped brace corresponding to the viewing space region 5111 can be positioned at a location that is offset from the center line between the first and second tool guides 510′.

In some embodiments, the first tool guide 510′ and second tool guide 510′ include proximal portions extending at a non-zero angle, for example an acute or obtuse angle, with respect to each other. For example, upper portions of the guides 510′ of the embodiment of FIG. 16C are at an acute angle with respect to each other. In some embodiments, the first tool guide 510′ and second tool guide 510′ include distal portions extending in a direction parallel to each other. For example, lower portions of the guides 510′ of the embodiment of FIG. 16C, that is the portions below bends 513 are parallel to each other. The angled relationship of the tool guides 510′ at their top portions allow for a broader area for tool access and insertion. The parallel relationship of the tool guides 510′ at their lower portions allow for an improved interface with the ports of the connector 250.

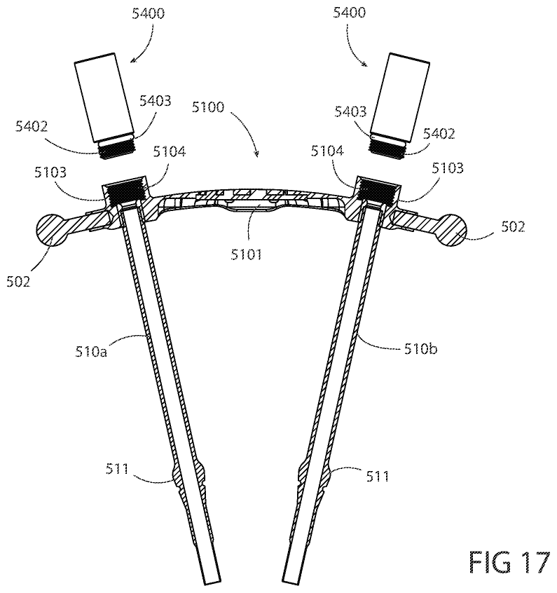

Referring additionally to FIG. 17, a sectional side view of a shaped instrument support is illustrated, consistent with the present inventive concepts. Instrument support 5100 comprises a brace 5101 comprising at least one threaded projection 5103 aligned with the position of a tool guide 510. Brace 5101 can comprise two threaded projections 5103, such that a single projection 5103 is aligned with a single tool guide 510 (tool guides 510a, 510b shown). Projection 5103 can comprise threads 5104 constructed and arranged to mate with a threaded adapter 5400. Adapters 5400 are configured to extend the length of tool guide 510, as shown in FIG. 17. Adapter 5400 can comprise threads 5402 constructed and arranged to mate with threads 5104 of the corresponding threaded projection 5103. Adapters 5400 can comprise different shapes, sizes, and lengths. In some embodiments, adapter 5400 comprises a seal assembly 530 (such as is shown in FIG. 8) constructed and arranged to maintain a seal. In some embodiments, adapter 5400 comprises an O-ring 5403 to prevent gas, or other fluid, from passing through the connection between threads 5402 of adapter 5400 and threads 5104 of projection 5103.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. A system for performing a medical procedure at a target location of a patient, comprising:
an articulating probe comprising at least a distal portion;
a feeder configured to advance, retract, and steer the articulating probe;
a first introducer attached to the feeder and configured to slidingly receive the articulating probe; and
a second introducer for providing access to an internal location of the patient;
wherein the first introducer operably attaches to the second introducer, such that the articulating probe can be advanced through the first and second introducers to the target location;
wherein the system further comprises at least one sealing element configured to maintain insufflation pressure at the target location.

2.- 229. (canceled)
